(19) United States
(12) Patent Application Publication
(10) Pub. No.: US 2023/0172921 A1
(43) Pub. Date: Jun. 8, 2023

(54) LERIGLITAZONE FOR TREATING LUNG INFLAMMATION AND INTERSTITIAL LUNG DISEASE

(71) Applicant: Minoryx Therapeutics S.L., Mataró, Barcelona (ES)

(72) Inventors: Marc MARTINELL PEDEMONTE, Mataró, Barcelona (ES); Maria Pilar PIZCUETA LALANZA, Mataró, Barcelona (ES); Anna VILALTA SAURA, Mataró, Barcelona (ES); Estefania TRAVER LÓPEZ, Mataró, Barcelona (ES); Sonia Maria POLI, Onex, Geneva (CH); Nuria IZQUIERDO USEROS, Badalona (ES)

(73) Assignee: Minoryx Therapeutics S.L., Mataró, Barcelona (ES)

(21) Appl. No.: 17/997,393

(22) PCT Filed: Apr. 30, 2021
(86) PCT No.: PCT/IB2021/053651

§ 371 Date: Oct. 28, 2022

(30) Foreign Application Priority Data
Apr. 30, 2020 (EP) 20382356.2

(51) Int. Cl. A61K 31/4439; A61P 31/14; A61P 11/00

(52) U.S. Cl. CPC A61K 31/4439; A61P 31/14; A61P 11/00

(57) ABSTRACT

The present disclosure provides methods of treating viral-induced inflammatory lung conditions or diseases, acute inflammation of the lung, or interstitial lung disease with 5-[[4-[2-[5-(1-hydroxyethyl)pyridin-2-yl]ethoxy]phenyl]methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof.

### BACKGROUND OF THE INVENTION

### Field of the Invention

The present disclosure provides methods of treating viral-induced inflammatory lung conditions or diseases, acute inflammation of the lung, or interstitial lung disease with 5-[[4-[2-[5-(1-hydroxyethyl)pyridin-2-yl]ethoxy]phenyl]methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof.

### Background

Lung disease is a significant health problem. For example, interstitial lung disease (ILD) is a large group of diseases that affect the tissue and spaces (interstitial) around the air sacs (alveoli) in the lung. Idiopathic pulmonary fibrosis (IPF) is the most common form of ILD. It is estimated that IPF affects 1 out of 200 adults over the age of 60 in the United States. Lung damage from an ILD may be irreversible. There exists a need for drugs to treat IPF and other ILDs.

Many lung diseases are associated with acute lung inflammation. Acute lung injury is characterized by hypoxia, inflammation, lung edema, decreased respiratory compliance, and can be regarded as a milder form or a precursor to the more aggressive inflammatory process acute respiratory distress syndrome (ARDS). Leukocyte recruitment is a key event in acute lung injury, leading to inflammation and plasma leakage.

For example, acute respiratory distress syndrome (ARDS) involves the rapid onset of progressive malfunction of the lungs, and is usually associated with the malfunction of other organs due to the inability to take up oxygen. This condition is associated with extensive lung inflammation and small blood vessel injury in all affected organs. Causes may include, but are not limited to, bacterial or viral infections, trauma, sepsis or aspiration.

Coronaviruses are positive-stranded RNA viruses with large genome sizes that are known to cause diseases in animals and in humans. In humans, coronaviruses cause respiratory tract infections that are typically mild, such as the common cold. But coronaviruses can also cause much more serious infections such coronavirus-induced severe acute respiratory syndrome (SARS). Woo et al., Microbiol. Immunol. 49:899-908 (2005). Seven strains of human coronaviruses are known: human coronavirus 229E (HCoV-229E); human coronavirus OC43 (HCoV-OC43); severe acute respiratory syndrome coronavirus (SARS-CoV); human coronavirus NL63 (HCoV-NL63, New Haven coronavirus); human coronavirus HKU1; Middle East respiratory syndrome-related coronavirus (MERS-CoV, also known as novel coronavirus 2012 and HCoV-EMC); and severe acute respiratory syndrome coronavirus 2 (SARS-CoV-2), also as 2019-nCoV or novel coronavirus 2019.

In March 2020, the World Health Organization declared the outbreak of coronavirus disease 2019 (COVID-19) a pandemic. Like the severe acute respiratory syndrome (SARS) outbreak of 2003, and the Middle East respiratory syndrome (MERS) outbreaks of 2012, 2015, and 2018, COVID-19 has caused serious illness and death around the world. There exists a need for drugs to treat the inflammatory lung conditions or diseases that are triggered by coronavirus and other viral infections. Li et al., J Med Virol 92:424-432 (2020).

Many viral infections including those caused by human coronaviruses, e.g., COVID-19, HIV, and influenza are associated with the development of ARDS and other inflammatory conditions or diseases. Mehta et al., Lancet 395:1033-1034 (2020); Huang et al., The Lancet. 395(10223):497-506 (2020). ARDS is triggered by injury to the alveolar-capillary barrier, resulting in fluid accumulation and acute respiratory failure. ARDS triggered in viral infections has been linked to dramatically increased host immune response and immune cell infiltration into the lung as well as cytokine expression which may lead to lung injury. Rockx et al., Journal of Virology 83: 7062-7074 (2009). Accumulating evidence suggests that a subgroup of patients with severe COVID-19 have a cytokine storm syndrome which resembles the secondary haemophagocytic lymphohistiocytosis (sHLH) commonly triggered by viral infections. This is a hyperinflammatory syndrome characterized by a fulminant and fatal hypercytokinaemia with multiorgan failure. The cytokine profile includes IL-2, IP-10, TNF-alpha, and IL-6. Very high levels of IL-6 were observed in infected patients by COVID in a recent study in China. Ruan et al., Intensive Care Med 2020; published online March 3. DOI:10.1007/s00134-020-05991-x.

5-[[4-[2-[5-(1-hydroxyethyl)pyridin-2-yl]ethoxy]phenyl]methyl]-1,3-thiazolidine-2,4-dione is a selective PPAR gamma agonist that can be used to treat inflammatory respiratory diseases such as chronic obstructive pulmonary disorder and viral diseases of the central nervous system such as meningitis. US 2020/0093812; US 2016/0235729.

### BRIEF SUMMARY OF THE INVENTION

In one aspect, the present disclosure provides methods of treating an inflammatory lung condition or disease caused by a viral infection, acute inflammation of the lung, or interstitial lung disease (ILD) in a subject in need thereof, the method comprising administering a therapeutically effective amount of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, to the subject

In another aspect, the present disclosure provides methods of treating an inflammatory lung condition or disease, e.g., a hyperinflammatory syndrome, hypercytokinaemia, ARDS, or haemophagocytic lymphohistiocytosis (sHLH), caused by a viral infection, e.g., a coronavirus, respiratory syncytial virus, influenza virus, adenovirus or HIV (human immunodeficiency) virus in a subject in need thereof, the method comprising administering a therapeutically effective amount of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, to the subject.

In another aspect, the present disclosure provides 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, for use in treating an inflammatory lung condition or disease caused by a viral infection in a subject in need thereof.

In another aspect, the present disclosure provides the use of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, for the manufacture of a medicament for treating an inflammatory lung condition or disease caused by a viral infection.

In another aspect, the present disclosure provides methods of treating acute inflammation of the lung in a subject in need thereof, the method comprising administering a therapeutically effective amount of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, to the subject.

In another aspect, the present disclosure provides 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, for use in treating acute inflammation of the lung in a subject in need thereof.

In another aspect, the present disclosure provides the use of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, for the manufacture of a medicament for treating acute inflammation of the lung.

In another aspect, the present disclosure provides methods of treating ILD in a subject in need thereof, the method comprising administering a therapeutically effective amount of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, to the subject.

In another aspect, the present disclosure provides 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, for use in treating ILD in a subject in need thereof.

In another aspect, the present disclosure provides the use of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or a therapeutically effective amount of 5-[[4-[2-[5-acetylpyridin-2-yl]ethoxy]phenyl]methyl]-1,3-thiazolidine-2,4-dione, or a pharmaceutically acceptable salt thereof, for the manufacture of a medicament for treating ILD.

In another aspect, the present disclosure provides a method for decreasing the level of one or more cytokines, e.g., IP-10, IL-4, IFNα2, IFNγ, TNFα, MCP1, IL-6, or MIP1-α, or a combination thereof, in a subject in need thereof, the method comprising administering to the subject a therapeutically effective amount of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof, wherein the subject has an inflammatory lung condition or disease caused by a viral infection, acute inflammation of the lung, or interstitial lung disease.

In another aspect, the present disclosure provides 5-[[4-[2-[5-(1-hydroxyethyl)pyridin-2-yl]ethoxy]phenyl]methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof, for use in decreasing the level of one or more cytokines, e.g., IP-10, IL-4, IFNα2, IFNγ, TNFα, MCP1, IL-6, or MIP1-α, or a combination thereof, in a subject in need thereof, wherein the subject has an inflammatory lung condition or disease caused by a viral infection, acute inflammation of the lung, or interstitial lung disease.

In another aspect, the present disclosure provides the use of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof, for the manufacture of a medicament for decreasing the level of one or more cytokines, e.g., IP-10, IL-4, IFNα2, IFNγ, TNFα, MCP1, IL-6, or MIP1-α, or a combination thereof, in a subject wherein the subject has an inflammatory lung condition or disease caused by a viral infection, acute inflammation of the lung, or interstitial lung disease.

In another aspect, the present disclosure provides a kit comprising 5-[[4-[2-[5-(1-hydroxyethyl)pyridin-2-yl]ethoxy]phenyl]methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof, and instructions for using 5-[[4-[2-[5-(1-hydroxyethyl)pyridin-2-yl]ethoxy]phenyl]methyl]-1,3-thiazolidine-2,4-dione, or a pharmaceutically acceptable salt thereof or 5-[[4-[2-[5-acetylpyridin-2-yl]ethoxy]phenyl]methyl]-1,3-thiazolidine-2,4-dione, or a pharmaceutically acceptable salt thereof, to treat a subject having an inflammatory lung condition or disease caused by a bacterial or viral infection, acute inflammation of the lung, or ILD.

### BRIEF DESCRIPTION OF THE DRAWINGS

FIG. 1 is a graph showing IP-10 released into the supernatant measured with Luminex technology 24 hours post-infection. Macrophages exposed or not to SARS-CoV-2 and cultured with the indicated concentrations of leriglitazone (“Leri”) at therapeutic conditions include duplicate measurements of three different donors. Statistical differences were assayed with a paired t test and considered significant for p values<0.005. * P<0.05; ** P<0.01; ***: P<0.001 and ****P<0.0001.

FIG. 2 is a graph showing IL-4 released into the supernatant measured with Luminex technology 24 hours post-infection. Macrophages exposed or not to SARS-CoV-2 and cultured with the indicated concentrations of leriglitazone (“Leri”) at therapeutic conditions include duplicate measurements of three different donors. Statistical differences were assayed with a paired t test and considered significant for p values<0.005. * P<0.05; ** P<0.01; ***: P<0.001 and ****P<0.0001.

FIG. 3 is a graph showing IFN2α released into the supernatant measured with Luminex technology 24 hours post-infection. Macrophages exposed or not to SARS-CoV-2 and cultured with the indicated concentrations of leriglitazone (“Leri”) at therapeutic conditions include duplicate measurements of three different donors. Statistical differences were assayed with a paired t test and considered significant for p values<0.005. * P<0.05; ** P<0.01; ***: P<0.001 and ****P<0.0001.

FIG. 4 is a graph showing IFNγ released into the supernatant measured with Luminex technology 24 hours post-infection. Macrophages exposed or not to SARS-CoV-2 and cultured with the indicated concentrations of leriglitazone (“Leri”) at therapeutic conditions include duplicate measurements of three different donors. Statistical differences were assayed with a paired t test and considered significant for p values<0.005. * P<0.05; ** P<0.01; ***: P<0.001 and ****P<0.0001.

FIG. 5 is a graph showing TNFα released into the supernatant measured with Luminex technology 24 hours post-infection. Macrophages exposed or not to SARS-CoV-2 and cultured with the indicated concentrations of leriglitazone (“Leri”) at therapeutic conditions include duplicate measurements of three different donors. Statistical differences were assayed with a paired t test and considered significant for p values<0.005. * P<0.05; ** P<0.01; ***: P<0.001 and ****P<0.0001.

FIG. 6 is a graph showing MCP1 released into the supernatant measured with Luminex technology 24 hours post-infection. Macrophages exposed or not to SARS-CoV-2 and cultured with the indicated concentrations of leriglitazone (“Leri”) at therapeutic conditions include duplicate measurements of three different donors. Statistical differences were assayed with a paired t test and considered significant for p values<0.005. * P<0.05; ** P<0.01; ***: P<0.001 and ****P<0.0001.

FIG. 7 is a graph showing IL-6 released into the supernatant were measured with Luminex technology 24 hours post-infection. Macrophages exposed or not to SARS-CoV-2 and cultured with the indicated concentrations of leriglitazone (“Leri”) at therapeutic conditions include duplicate measurements of three different donors. Statistical differences were assayed with a paired t test and considered significant for p values<0.005. * P<0.05; ** P<0.01; ***: P<0.001 and ****P<0.0001.

FIG. 8 is a graph showing MIP1-α released into the supernatant were measured with Luminex technology 24 hours post-infection. Macrophages exposed or not to SARS-CoV-2 and cultured with the indicated concentrations of leriglitazone (“Leri”) at therapeutic conditions include duplicate measurements of three different donors. Statistical differences were assayed with a paired t test and considered significant for p values<0.005. * P<0.05; ** P<0.01; ***: P<0.001 and ****P<0.0001.

FIG. 9 is a graph showing IL-2 released into the supernatant were measured with Luminex technology 24 hours post-infection. Macrophages exposed or not to SARS-CoV-2 and cultured with the indicated concentrations of leriglitazone (“Leri”) at therapeutic conditions include duplicate measurements of three different donors. Statistical differences were assayed with a paired t test and considered significant for p values<0.005. * P<0.05; ** P<0.01; ***: P<0.001 and ****P<0.0001.

FIG. 10 is an illustration of the study schematic used in the LPS induced acute lung injury model in mice.

FIG. 11 is a bar graph showing the amount of CD45⁺ cells in bronchoalveolar lavage fluid (BALF) from the LPS induced acute lung injury (ALI) model following administration of MIN-102 or M3 at the indicated doses.

FIG. 12 is a bar graph showing the amount neutrophils in BALF from the LPS induced ALI model following administration of MIN-102 or M3 at the indicated doses.

FIG. 13 is a bar graph showing the amount T-cells in BALF from the LPS induced ALI model following administration of MIN-102 or M3 at the indicated doses.

FIG. 14 is a bar graph showing the amount of total protein in BALF from the LPS induced ALI model following administration of MIN-102 or M3 at the indicated doses.

FIG. 15 is a bar graph showing the amount of IL-6 in BALF from the LPS induced ALI model following administration of MIN-102 or M3 at the indicated doses.

FIG. 16 is a bar graph showing the amount of IL-12p40 in BALF from the LPS induced ALI model following administration of MIN-102 or M3 at the indicated doses.

FIG. 17 is a bar graph showing the amount of CXCL10/IP-10 BALF from the LPS induced ALI model following administration of MIN-102 or M3 at the indicated doses.

FIG. 18 is a bar graph showing the amount of CXCL 9 in BALF from the LPS induced ALI model following administration of MIN-102 or M3 at the indicated doses.

FIG. 19 is a bar graph showing the amount RANTES BALF from the LPS induced ALI model following administration of MIN-102 or M3 at the indicated doses.

FIG. 20 is a bar graph showing the amount of TNFα in BALF from the LPS induced ALI model following administration of MIN-102 or M3 at the indicated doses.

FIG. 21 is a bar graph showing the mean histopathology score the LPS induced ALI model following administration of MIN-102 or M3 at the indicated doses with respect to mononuclear cell infiltration in the perivascular/peribronchiolar space and neutrophil recruitment in the interstitium and the alveoli.

FIG. 22 is a bar graph showing the mean histopathology score the LPS induced ALI model following administration of MIN-102 or M3 at the indicated doses with respect to septal thickening in the alveoli. Statistical differences vs. vehicle group were assayed with a One-way ANOVA with Dunnet post-test and considered significant for p values<0.005. * P<0.05; ** P<0.01; ***: P<0.001 and ****P<0.0001.

### DETAILED DESCRIPTION OF THE INVENTION

Anti-inflammatory agents have failed to show any mortality benefit in ARDS in human subjects. See Han and Mallampalli, J. Immunol. 194:855-860 (2015). Also, pioglitazone, a peroxisome proliferator-activated receptor-7 (PPAR-7) agonist, had no effect on endotoxin-induced lung inflammation in healthy human volunteers. Chen et al., PLoS ONE 13(2):e0191783. https://doi.org/10.1371/journal.pone.0191783 (2018). Applicant unexpectedly discovered that leriglitazone can be used to treat coronavirus-induced inflammatory lung conditions or diseases such as ARDS or hypercytokinaemia in a subject. Without wishing to be bound by any particular theory, this discovery is based at least in part on leriglitazone's unexpected distribution into the lung tissue of a subject and the amount of unbound drug in plasma, see Table 1, to cause a decrease in, e.g., IL-6, IP-10, IL-4, IFNα2, IFNγ, MCP1, and TNF-alpha cytokine levels without affecting lymphocyte number and function. Leriglitazone also inhibits leukocyte infiltration and septal alveoli alteration produced by acute lung injury.

TABLE 1

| | Lung/Blood Distribution Ratio | Unbound % in Plasma |
|---|---|---|
| Drug | (rat) | (human) |
| Leriglitazone | 0.83 | 3.5% |
| Pioglitazone | 0.3 | 0.2% |

### I. Compounds of the Disclosure

The term “Compound 1” as used herein refers, collectively, to 5-[[4-[2-[5-(1-hydroxyethyl)pyridin-2-yl]ethoxy]phenyl]methyl]-1,3-thiazolidine-2,4-dione, all possible stereoisomers, e.g., enantiomers and diastereomers, and mixtures, e.g., racemic mixtures, thereof, and the pharmaceutically acceptable salts thereof. In one embodiment, Compound 1 is administered to the subject. The structure of 5-[[4-[2-[5-(1-hydroxyethyl)pyridin-2-yl]ethoxy]phenyl]methyl]-1,3-thiazolidine-2,4-dione is:

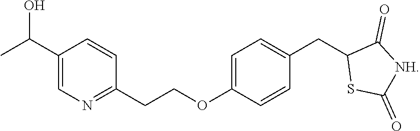 [SMILES: CC(O)c1ccc(CCOc2ccc(CC3SC(=O)NC3=O)cc2)nc1]

The present disclosure encompasses the use of stereoisomers of 5-[[4-[2-[5-(1-hydroxyethyl)pyridin-2-yl]ethoxy]phenyl]methyl]-1,3-thiazolidine-2,4-dione. 5-[[4-[2-[5-(1-hydroxyethyl)pyridin-2-yl]ethoxy]phenyl]methyl]-1,3-thiazolidine-2,4-dione has two asymmetric centers and thus four stereoisomers are possible as follows:

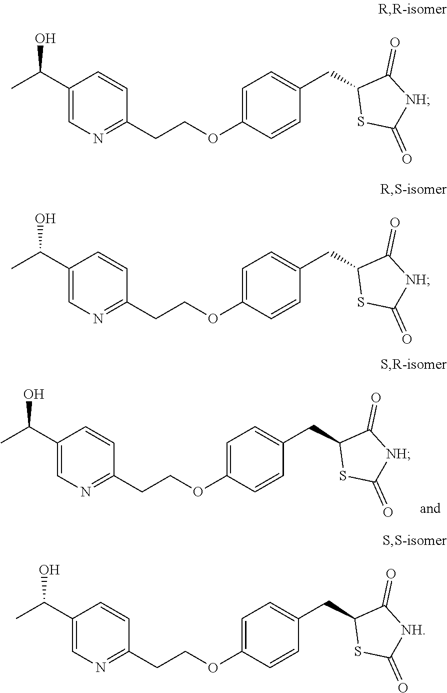 [SMILES: C[C@@H](O)c1ccc(CCOc2ccc(C[C@H]3SC(=O)NC3=O)cc2)nc1 ; C[C@H](O)c1ccc(CCOc2ccc(C[C@H]3SC(=O)NC3=O)cc2)nc1 ; C[C@@H](O)c1ccc(CCOc2ccc(C[C@@H]3SC(=O)NC3=O)cc2)nc1 ; C[C@H](O)c1ccc(CCOc2ccc(C[C@@H]3SC(=O)NC3=O)cc2)nc1]

In one embodiment, one of the four stereoisomers of 5-[[4-[2-[5-(1-hydroxyethyl)pyridin-2-yl]ethoxy]phenyl]methyl]-1,3-thiazolidine-2,4-dione, or a pharmaceutically acceptable salt thereof, is administered to the subject.

In another embodiment, a mixture comprising two of the four stereoisomers of 5-[[4-[2-[5-(1-hydroxyethyl)pyridin-2-yl]ethoxy]phenyl]methyl]-1,3-thiazolidine-2,4-dione, or the pharmaceutically acceptable salts thereof, is administered to the subject. The two stereoisomers of the mixture can be present in equimolar amounts, or one stereoisomer of the mixture is present in a minor amount, e.g., less than 10 wt. %, less than 3 wt. %, less than 1 wt. %, or less than 0.1 wt. % as compared to the other stereoisomer.

In another embodiment, a mixture comprising three of the four stereoisomers of 5-[[4-[2-[5-(1-hydroxyethyl)pyridin-2-yl]ethoxy]phenyl]methyl]-1,3-thiazolidine-2,4-dione, or the pharmaceutically acceptable salts thereof, is administered to the subject. The three stereoisomers of the mixture can be present in equimolar amounts; or one stereoisomer of the mixture is present in a minor amount, e.g., less than 10 wt. %, less than 3 wt. %, less than 1 wt. %, or less than 0.1 wt. % as compared to the other two stereoisomers; or two stereoisomers of the mixture are present in a minor amount, e.g., less than 10 wt. %, less than 3 wt. %, less than 1 wt. %, or less than 0.1 wt. % as compared to the other stereoisomer.

In another embodiment, a mixture comprising all four stereoisomers of 5-[[4-[2-[5-(1-hydroxyethyl)pyridin-2-yl]ethoxy]phenyl]methyl]-1,3-thiazolidine-2,4-dione, or the pharmaceutically acceptable salts thereof, is administered to the subject. In another embodiment, the mixture comprises each stereoisomer in an amount of 20%±10% w/w. In another embodiment, the mixture comprises each stereoisomer in an amount of 25%±5% w/w. In another embodiment, the mixture comprises each stereoisomer in an amount of 25% w/w. In another embodiment, one stereoisomer of the mixture is present in a minor amount, e.g., less than 10 wt. %, less than 3 wt. %, less than 1 wt. %, or less than 0.1 wt. % as compared to the other three stereoisomers. In another embodiment, two stereoisomers of the mixture are present in a minor amount, e.g., less than 10 wt. %, less than 3 wt. %, less than 1 wt. %, or less than 0.1 wt. % as compared to the other two stereoisomers. In another embodiment, three stereoisomers of the mixture are present in a minor amount, e.g., less than 10 wt. %, less than 3 wt. %, less than 1 wt. %, or less than 0.1 wt. % as compared to the other stereoisomer.

The term “Compound 2” as used herein refers, collectively, to 5-[[4-[2-[5-acetylpyridin-2-yl]ethoxy]phenyl]methyl]-1,3-thiazolidine-2,4-dione, all possible stereoisomers, e.g., the R- and S-enantiomers, and mixtures, e.g., racemic mixtures, thereof, and the pharmaceutically acceptable salts thereof. In one embodiment, Compound 2 is administered to the subject. The structure of 5-[[4-[2-[5-acetylpyridin-2-yl]ethoxy]phenyl]methyl]-1,3-thiazolidine-2,4-dione is:

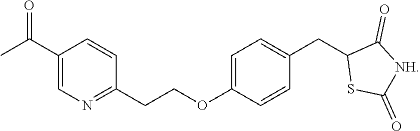 [SMILES: CC(=O)c1ccc(CCOc2ccc(CC3SC(=O)NC3=O)cc2)nc1]

5-[[4-[2-[5-Acetylpyridin-2-yl]ethoxy]phenyl]methyl]-1,3-thiazolidine-2,4-dione is metabolized to give 5-[[4-[2-[5-(1-hydroxyethyl)pyridin-2-yl]ethoxy]phenyl]methyl]-1,3-thiazolidine-2,4-dione. WO 2019/234690. 5-[[4-[2-[5-Acetylpyridin-2-yl]ethoxy]phenyl]methyl]-1,3-thiazolidine-2,4-dione is also known as M3.

In the therapeutic methods and uses disclosed herein, Compound 1, Compound 2, or a mixture of Compound 1 and Compound 2 can be administered to a subject having an inflammatory lung condition or disease caused by a viral infection, acute inflammation of the lung, or interstitial lung disease.

The present disclosure encompasses the use of salts of Compound 1 and Compound 2 including, but not limited to, the hydrochloride salt. The term “pharmaceutically acceptable salt” as used herein, refers to any salt, e.g., obtained by reaction of Compound 1 or Compound 2 with an acid that is physiologically tolerated in the subject, e.g., a human. Salts of Compound 1 or Compound 2 may be derived from inorganic or organic acids. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like.

In one embodiment, Compound 1 is the hydrochloride salt of racemic 5-[[4-[2-[5-(1-hydroxyethyl)pyridin-2-yl]ethoxy]phenyl]methyl]-1,3-thiazolidine-2,4-dione. The hydrochloride salt of racemic 5-[[4-[2-[5-(1-hydroxyethyl)pyridin-2-yl]ethoxy]phenyl]methyl]-1,3-thiazolidine-2,4-dione is also known as 5-[[4-[2-[5-(1-hydroxyethyl)pyridin-2-yl]ethoxy]phenyl]methyl]-1,3-thiazolidine-2,4-dione hydrochloride, MIN-102, or leriglitazone.

In one embodiment, Compound 2 is the hydrochloride salt of racemic 5-[[4-[2-[5-acetylpyridin-2-yl]ethoxy]phenyl]methyl]-1,3-thiazolidine-2,4-dione.

The present disclosure encompasses the use of solvates of Compound 1 or Compound 2. Solvates typically do not significantly alter the physiological activity or toxicity of a compound, and as such may function as pharmacological equivalents. The term “solvate” as used herein is a combination, physical association and/or solvation of Compound 1 or Compound 2 with a solvent molecule such as, e.g., a disolvate, monosolvate or hemisolvate, where the ratio of solvent molecule to Compound 1 or Compound 2 is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compound 1 or Compound 2 can be present as solvated forms with a pharmaceutically acceptable solvent, such as water, methanol, ethanol, and the like, and it is intended that the disclosure includes both solvated and unsolvated forms of Compound 1 or Compound 2. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate involves dissolving Compound 1 or Compound 2 in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### II. Methods of the Disclosure

Compound 1, or a pharmaceutical composition thereof, or Compound 2, or a pharmaceutical composition thereof, can be administered to any subject in need thereof, e.g., a subject already suffering from an inflammatory lung condition or disease caused by a viral infection, a subject suspected of having an inflammatory lung condition or disease caused by a viral infection, or a subject at risk of developing an inflammatory lung condition or disease affecting the lung caused by a viral infection.

In one embodiment, the disclosure provides a method of treating an inflammatory lung condition or disease caused by a viral infection in a subject in need thereof, the method comprising administering a therapeutically effective amount of Compound 1 or Compound 2 to the subject.

In another embodiment, the disclosure provides a method of treating an inflammatory lung condition or disease caused by a viral infection in a subject in need thereof, the method comprising administering a therapeutically effective amount of a mixture of Compound 1 and Compound 2 to the subject.

In another embodiment, the disclosure provides Compound 1, or a pharmaceutical composition thereof, or Compound 2, or a pharmaceutical composition thereof, for use in treating an inflammatory lung condition or disease caused by a viral infection.

In another embodiment, the disclosure provides the use of Compound 1 or Compound 2 in the manufacture of a medicament for treating an inflammatory lung condition or disease caused by a viral infection.

In another embodiment, the disclosure provides methods of treating acute inflammation of the lung, e.g., pneumonia, ARDS, the method comprising administering a therapeutically effective amount of Compound 1 or Compound 2 to the subject.

In another embodiment, the disclosure provides methods of treating acute inflammatory episodes (AIEs) in chronic pathologies, e.g., in chronic obstructive pulmonary disease (COPD), the method comprising administering a therapeutically effective amount of Compound 1 or Compound 2 to the subject.

In another embodiment, the present disclosure provides Compound 1 or Compound 2, or a pharmaceutical composition thereof, for use in treating acute inflammation of the lung in a subject in need thereof.

In another embodiment, the disclosure provides Compound 1 or Compound 2, or a pharmaceutical composition thereof, for use in treating AIEs in chronic pathologies, e.g., in COPD.

In another embodiment, the present disclosure provides the use of Compound 1 or Compound 2, for the manufacture of a medicament for treating acute inflammation of the lung.

In another embodiment, the disclosure provides the use of Compound 1 or Compound 2, or a pharmaceutical composition thereof, for the manufacture of a medicament for treating AIEs in chronic pathologies, e.g., in COPD.

In another embodiment, the present disclosure provides methods of treating ILD, e.g., IPF, in a subject in need thereof, the method comprising administering a therapeutically effective amount of Compound 1 or Compound 2 to the subject.

In another embodiment, the present disclosure provides Compound 1 or Compound 2, or a pharmaceutical composition thereof, for use in treating ILD in a subject in need thereof.

In another embodiment, the present disclosure provides the use of Compound 1 or Compound 2 for the manufacture of a medicament for treating ILD.

In some embodiments, methods of the present disclosure involve detecting cytokine expression levels in a subject or a sample obtained from a subject.

In one embodiment, the level of one or more cytokines, e.g., IP-10, IL-4, IFNα2, IFNγ, TNFα, MCP1, IL-6, or MIP1-α, or a combination thereof, is higher in a subject of one phenotypic status, e.g., a subject having an inflammatory lung condition or disease caused by a viral infection, acute inflammation of the lung, or interstitial lung disease, as compared to a subject of another phenotypic status, e.g., a healthy subject.

In another embodiment, the disclosure provides a method for decreasing inflammatory cells, cytokines, or chemokines, or a combination thereof, in a subject in need thereof, the method comprising administering to the subject a therapeutically effective amount of Compound 1 or Compound 2. In another embodiment, the subject has an inflammatory lung condition or disease caused by a viral infection, acute inflammation of the lung, or interstitial lung disease. In another embodiment, the subject has an acute inflammation of the lung caused by a bacterial infection. In another embodiment, the subject has ARDS.

In another embodiment, the disclosure provides a method for decreasing the level of IP-10, IL-4, IFNα2, IFNγ, TNFα, MCP1, IL-6, or MIP1-α, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, the disclosure provides a method for decreasing the level of leukocytes, e.g., CD45⁺ cells (pan leukocyte marker), neutrophils, or T cells, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, the disclosure provides a method for decreasing the level of IL-12p40, CXCL10/IP-10, CXCL 9, or RANTES, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, the disclosure provides a method for decreasing the level of IP-10, IL-4, IFNα2, IFNγ, TNFα, MCP1, IL-6, MIP1-α, IL-12p40, CXCL 9, RANTES, CD45⁺ cells, T cells, or neutrophils, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, the present disclosure provides Compound 1 or Compound 2 for use in decreasing the level of one or more cytokines, e.g., IP-10, IL-4, IFNα2, IFNγ, TNFα, MCP1, IL-6, or MIP1-α, or a combination thereof, in a subject in need thereof, wherein the subject has an inflammatory lung condition or disease caused by a viral infection, acute inflammation of the lung, or interstitial lung disease.

In another embodiment, the disclosure provides Compound 1 or Compound 2 for use in decreasing the level of leukocytes, e.g., CD45⁺ cells (pan leukocyte marker), neutrophils, or T cells, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, the disclosure provides Compound 1 or Compound 2 for use in decreasing the level of IL-12p40, CXCL10/IP-10, CXCL 9, or RANTES, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, the disclosure provides Compound 1 or Compound 2 for use in decreasing the level of IP-10, IL-4, IFNα2, IFNγ, TNFα, MCP1, IL-6, MIP1-α, IL-12p40, CXCL10/IP-10, CXCL 9, RANTES, CD45⁺ cells, neutrophils, or T cells, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, the present disclosure provides the use of Compound 1 or Compound for the manufacture of a medicament for decreasing the level of one or more cytokines, e.g., IP-10, IL-4, IFNα2, IFNγ, TNFα, MCP1, IL-6, or MIP1-α, or a combination thereof, in a subject wherein the subject has an inflammatory lung condition or disease caused by a viral infection, acute inflammation of the lung, or interstitial lung disease.

In another embodiment, the disclosure provides the use of Compound 1 or Compound for the manufacture of a medicament for decreasing the level of leukocytes, e.g., CD45⁺ cells, neutrophils, or T cells, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, the disclosure provides the use of Compound 1 or Compound for the manufacture of a medicament for decreasing the level of IL-12p40, CXCL10/IP-10, CXCL 9, or RANTES, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, the disclosure provides the use of Compound 1 or Compound for the manufacture of a medicament for decreasing the level of IP-10, IL-4, IFNα2, IFNγ, TNFα, MCP1, IL-6, MIP1-α, IL-12p40, CXCL10/IP-10, CXCL 9, RANTES, CD45⁺ cells, T cells, or neutrophils, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

In another embodiment, Compound 1 is the hydrochloride salt of 5-[[4-[2-[5-(1-hydroxyethyl)pyridin-2-yl]ethoxy]phenyl]methyl]-1,3-thiazolidine-2,4-dione (i.e., leriglitazone).

In another embodiment, the inflammatory lung condition or disease caused by a viral infection is hyperinflammation.

In another embodiment, the inflammatory lung condition or disease caused by a viral infection is systemic inflammatory response syndrome (SIRS).

In another embodiment, the inflammatory lung condition or disease caused by a viral infection is acute respiratory distress syndrome (ARDS) or acute lung injury (ALI).

In another embodiment, the inflammatory lung condition or disease caused by a viral infection is pneumonia.

In another embodiment, the inflammatory lung condition or disease caused by a viral infection is hyperinflammatory syndrome. In another embodiment, the hyperinflammatory syndrome is hypercytokinaemia or “cytokine storm.” In another embodiment, the hypercytokinaemia is associated with multiorgan failure. In another embodiment, the hyperinflammatory syndrome is haemophagocytic lymphohistiocytosis.

In another embodiment, the viral infection is caused by a double and single stranded DNA virus. Exemplary DNA viruses include, but are not limited to, chickenpox, human cytomegalovirus, herpes simplex virus type 1, adenovirus, papillovirus, varicell-zoster, cytomegalovirus, Epstein-Barr, smallpox, cow pox, vaccionia virus and parvovirus.

In another embodiment, the viral infection is caused by a RNA virus. Exemplary RNA viruses include, but are not limited to, coronavirus, respiratory syncytial virus, parainfluenza-3 virus, bovine viral diarrhea virus, Venezuelan equine encephalomyelitis virus, Dengue virus, yellow fever virus, Coxsackie B3 virus, encephalomyocarditis virus, influenza A virus, Zika virus, Ebola virus, Junin virus, Lassa Fever virus, Chikungunya virus reovirus, rotavirus, enterovirus, rhinovirus, hepatovirus, cardiovirus, aphthovirus, poliovirus, parechovirus, erbovirus, kobuvirus, teschovirus, coxsackie, Rubella virus, hepatitis C virus, Influenza virus A, influenzavirus B, influenzavirus C, isavirus, thogotovirus Measles virus, mumps virus, respiratory syncytial virus, Rinderpest virus, canine distemper virus, Rabies virus, Vesicular stomatitis, Marburg virus, hepatitis E virus, lentivirus (HIV), and hantavirus.

In another embodiment, the viral infection is caused by a reverse transcribing viruses such as HIV, caulimovirus, Cacao swollen-shoot virus (CSSV) and hepatitis B virus.

In another embodiment, the viral infection is caused by coronavirus.

In another embodiment, the coronavirus is an animal coronavirus.

In another embodiment, the coronavirus is a human coronavirus.

In another embodiment, the human coronavirus is HCoV-229E, HCoV-OC43, HCoV-NL63, HCoV-HKU1, SARS-CoV, MERS-CoV, or SARS-CoV-2, or a mutated strain of HCoV-229E, HCoV-OC43, HCoV-NL63, HCoV-HKU1, SARS-CoV, MERS-CoV, or SARS-CoV-2.

In another embodiment, the human coronavirus is HCoV-229E, HCoV-OC43, HCoV-NL63, HCoV-HKU1, SARS-CoV, MERS-CoV, or SARS-CoV-2.

In another embodiment, the human coronavirus is a mutated strain of HCoV-229E, HCoV-OC43, HCoV-NL63, HCoV-HKU1, SARS-CoV, MERS-CoV, or SARS-CoV-2.

In another embodiment, the human coronavirus is HCoV-229E.

In another embodiment, the human coronavirus is mutated strain of HCoV-229E.

In another embodiment, the human coronavirus is HCoV-OC43.

In another embodiment, the human coronavirus is mutated strain of HCoV-OC43.

In another embodiment, the human coronavirus is HCoV-NL63.

In another embodiment, the human coronavirus is mutated strain of HCoV-NL63.

In another embodiment, the human coronavirus is HCoV-HKU1.

In another embodiment, the human coronavirus is mutated strain of HCoV-HKU1.

In another embodiment, the human coronavirus is SARS-CoV.

In another embodiment, the human coronavirus is mutated strain of SARS-CoV.

In another embodiment, the human coronavirus is MERS-CoV.

In another embodiment, the human coronavirus is mutated strain of MERS-CoV.

In another embodiment, the human coronavirus is SARS-CoV-2.

In another embodiment, the human coronavirus is mutated strain of SARS-CoV-2.

In another embodiment, the acute inflammation of the lung is caused by a bacterial infection, a parasitic infection, any of the above-mentioned viral infections, Moldoveanu et al., J Inflamm Res 2:1-11 (2009), or any other cause. Rezoagli et al., Ann Transl Med 5(14):282 doi: 10.21037/atm.2017.06.62 (2017).

In another embodiment, the acute inflammation of the lung is pneumonia.

In another embodiment, the acute inflammation of the lung is ARDS. In another embodiment, the ARDS is not caused by a viral infection.

In another embodiment, the ILD is caused by drugs/chemicals, e.g., chemotherapy, environmental exposure; autoimmune disease; or any other, e.g., idiopathic, cause.

In another embodiment, the ILD is acute interstitial pneumonia, allergic bronchopulmonary aspergillosis, asbestosis, beryllium disease, autoimmune pulmonary alveolar proteinosis, Blau syndrome, bronchiolitis obliterans, bronchiolitis obliterans organizing pneumonia, chronic granulomatous disease, coal worker's pneumoconiosis, CREST syndrome, cryptogenic organizing pneumonia, cystic fibrosis, diffuse idiopathic pulmonary neuroendocrine cell hyperplasia, diffuse panbronchiolitis, fibrosing mediastinitis, Froster-Huch syndrome, idiopathic acute eosinophilic pneumonia, idiopathic pulmonary fibrosis (IPF), idiopathic pulmonary hemosiderosis, Kabuki syndrome, Kaolin pneumoconiosis, Kartagener syndrome, lung agenesis, Manouvrier syndrome, meconium aspiration syndrome, nontuberculous mycobacterial lung disease, pleuroparenchymal fibroelastosis, pulmonary alveolar microlithiasis, recurrent respiratory papillomatosis, respiratory distress syndrome, silicosis, tracheobronchomalacia, Wolf-Hirschhorn syndrome, or Young syndrome.

In another embodiment, the ILD is idiopathic pulmonary fibrosis (IPF).

In one embodiment, Compound 1 or Compound 2 is administered to a subject having an inflammatory lung condition or disease caused by a viral infection, acute inflammation of the lung, or ILD as a single agent. In another embodiment, Compound 1 or Compound 2 is administered to a subject having an inflammatory lung condition or disease caused by a viral infection, acute inflammation of the lung, or ILD in combination with one or more optional therapeutic agents. Optional therapeutic agents include hydroxychloroquine, choloroquine, antiretro viral agents such as Remdesivir and Favipiravir, IL6 inhibitors such as Kevzara and Actemra (Roche), corticoids, and anticytokine inhibitors including anakinra and anti jak inhibitors.

In another embodiment, Compound 1 or Compound 2 is administered to a subject having an inflammatory lung condition or disease caused by a viral infection, acute inflammation of the lung, or ILD in combination with one optional therapeutic agent.

In another embodiment, Compound 1 or Compound 2 is administered to a subject having an inflammatory lung condition or disease caused by a viral infection, acute inflammation of the lung, or ILD in combination with two optional therapeutic agents.

In another embodiment, Compound 1 or Compound 2 is administered to a subject having an inflammatory lung condition or disease caused by a viral infection, acute inflammation of the lung, or ILD in combination with three optional therapeutic agents.

Optional therapeutic agents include, but are not limited to, antiviral agents, antibiotic agents, and antifungal agents.

Non-limiting exemplary antiviral agents include oseltamivir, ganciclovir, lopinavir/ritonavir (Kaletra®), and remdesivir. Antiviral agents also include reverse transcriptase inhibitors (RTIs). In one embodiment, the RTI is a nucleoside reverse transcriptase inhibitors (NRTI). Non-limiting exemplary NRTIs include abacavir (ZIAGEN™), abacavir/lamivudine (Epzicom), abacavir/lamivudine/zidovudine (TRIZIVIR™), adefovir, alovudine, amdoxovir, apricitabine, ATRIPLA®, BARACLUDE®, BIKTARVY®, censavudine, COVIRACIL™, DAPD/DXG, D-D4FC, dexelvucitabine, didanosine (VIDEX™), didanosine extended-release (Videx EC), dOTC, EFdA, emtricitabine (EMTRIVA™), emtricitabine/tenofovir alafenamide (DESCOVY®), emtricitabine/tenofovir disoproxil fumarate (TRUVADA®), elvucitabine, fosalvudine, lamivudine/zidovudine (COMBIVIR™), EVIPLERA™, GENVOYA®, HMD™ KIVEXA™, lamivudine (EPIVIR™), LODENOSINE™, ODEFSEY®, PREVEON®, racivir, stampidine, stavudine (ZERIT™), STRIBILD®, TENOFOVIR™, tenofovir disoproxil fumarate (VIREAD™), TRIUMEQ®, Trizivir, VEMLIDY®, and zidovudine (RETROVIR™). In another embodiment, the RTI is a non-nucleoside reverse transcriptase inhibitor (NNRTI). Non-limiting exemplary NNRTIs include delavirdine, efavirenz, etravirine, nevirapine, and rilvipirine. Antiviral agents also include protease inhibitors. Non-limiting exemplary protease inhibitors include amprenavir, fosamprenavir, indinavir, nelfinavir, saquinavir, atazanavir, darunavir, and tipranavir.

In one embodiment, the one or more optional therapeutic agents comprise Merimepodib, Tocilizumab (Actemra®), Favipiravir (Avigan®), Tocilizumab/favipiravir, Leronlimab (PRO 140), Remdesivir, Ruxolitinib (Kevzara®), Sarilumab, Chloroquine phosphate (Aralen®, Resochin®), Chloroquine hydrochloride, Azithromycin (Zithromax®), hydroxychloroquine sulfate/azithromycin, Lopinavir/Ritonavir (Kaletra®), Eculizumab (Soliris®), Human monoclonal antibody targeting SARS-CoV-2, APNO1, Danoprevir (Ganovo®), TJM2 (TJ003234), Selinexor (XPOVIO®), Remestemcel-L (RYONCIL™), LAM-002 (apilimod), Rintatolimod (Ampligen®), DAS181, CM4620-IE, CAP-1002, SAB-185, ENU200, Camostat mesylate, IFX-1, Namilumab (IZN-101), GIAPREZA™ (angiotensin II), MN-166 (ibudilast), Rebif® (interferon beta-la), Ivermectin (Stromectol®, Mectizan®), NVX-CoV2373, Thiolanox®, Plitidepsin (Aplidin®), Opaganib (Yeliva®), RHB-107, Opaganib/RHB-107, EIDD-2801, Gimsilumab, TAK-888, ARMS-1, GENOSYL® (nitric oxide) gas, INOpulse®, BPI-002, rhu-pGSN, Galidesivir (BCX4430), BXT-10, L-glutamine oral powder (Endari®), Sylvant (siltuximab), Linebacker, Equivir, HTCC (N-(2-hydroxypropyl)-3-trimethylammonium 47 chitosan chloride), Darunavir (Prezista®), Darunavir/cobicistat (Prezcobix™), INOmax® (nitric oxide), WP1122, OYA1, Arbidol (umifenovir), Remescor®, MAN-01, STI-4920 (CMAB020), TZLS-501, IFN-alpha2b, Niclosamide, KL4, or WP1122.

Compound 1 or Compound 2 and the one or more optional therapeutic agents can be administered in combination under one or more of the following conditions: at different periodicities, at different durations, at different concentrations, by different administration routes, etc.

In one embodiment, Compound 1 or Compound 2 is administered to the subject according to a continuous dosing schedule, e.g., one or two times a day, every day for the duration of the treatment cycle.

In one embodiment, Compound 1 or Compound 2 is administered to the subject according to an intermittent dosing schedule, e.g., one or two times a day on Monday, Wednesday, or Friday, or any other non-continuous dosing schedule for the duration of the treatment cycle.

In one embodiment, Compound 1 or Compound 2 is administered to the subject orally. In another embodiment, the oral dosage form is an oral solution or an oral suspension.

The therapeutic methods provided herein comprise administering Compound 1 or Compound 2 to a subject having an inflammatory lung condition or disease caused by a viral infection, acute inflammation of the lung, or ILD in an amount which is effective to achieve its intended purpose. While individual needs vary, the determination of optimal ranges of effective amounts of each component is within the skill of the art. Typically, Compound 1 or Compound 2 is administered in an amount from about 0.05 mg/kg to about 500 mg/kg, about 0.05 mg/kg to about 100 mg/kg, about 0.05 mg/kg to about 50 mg/kg, or about 0.05 mg/kg to about 10 mg/kg. In one embodiment, Compound 1 or Compound 2 is administered once a day. In one embodiment, Compound 1 or Compound 2 is administered twice a day. These dosages are exemplary, but there can be individual instances in which higher or lower dosages are merited, and such are within the scope of this disclosure. In practice, the physician determines the actual dosing regimen that is most suitable for an individual subject, which can vary with the age, weight, and response of the particular subject.

The unit dose may comprise from about 0.01 to about 1000 mg, e.g., about 10 to about 300 mg, e.g., about 50 to about 200 mg of Compound 1 or Compound 2. In one embodiment, the unit oral dose of Compound 1 or Compound 2 is 0.05 mg, 1 mg, 3 mg, 5 mg, 7 mg, 9 mg, 10 mg 12 mg, 14 mg, 15 mg, 17 mg, 20 mg, 22 mg, 25 mg, 27 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, or about 200 mg. The unit dose may be administered one or more times daily. The unit does may be administered by any suitable route, e.g., orally, to the subject. In one embodiment, the unit dose is administered to the subject as an oral suspension.

A typical daily oral dosage of Compound 1 or Compound 2 is from 0.1 to 1000 mg, e.g. from 50 to 600 mg, e.g., from 80 to 300 mg, e.g., from 150 to 200 mg for an adult. In one embodiment, the daily dose for an adult is from about 50 mg to about 300 mg. In one embodiment, the daily dose for an adult is about 90 mg, 120 mg, 150 mg, 180 mg, or about 210 mg. In one embodiment, the daily dose for an adult is 180 mg administered as an oral suspension. A daily dose for a child is from about 0.1 mg to about 180 mg. In another embodiment, the daily oral dose for a child is from about 10 mg to about 100 mg.

Compound 1 or Compound 2 can be administered to a subject in the form of a raw chemical. Compound 1 or Compound 2 can also be administered to a subject as part of a pharmaceutical composition containing the compound combined with a suitable pharmaceutically acceptable carrier. Such a carrier can be selected from pharmaceutically acceptable excipients, vehicles, and auxiliaries. The term “pharmaceutically acceptable carrier,” “pharmaceutically acceptable vehicle,” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, Pa., 19th ed. 1995.

A pharmaceutical composition comprising Compound 1 or Compound 2 can contain from about 0.01 to 99 percent by weight, e.g., from about 0.25 to 75 percent by weight, of Compound 1 or Compound 2, e.g., about 1%, about 5%, about 10%, about 15%, about 20%, about 25%, about 30%, about 35%, about 40%, about 45%, about 50%, about 55%, about 60%, about 65%, about 70%, or about 75% by weight of Compound 1 or Compound 2.

In one embodiment, about 1 to 30 milliliters, e.g., about 5 mL, about 10 mL, about 15 mL, about 20 mL, or about 25 mL, of an oral suspension comprising about 15 mg of 5-[[4-[2-[5-(1-hydroxyethyl)pyridin-2-yl]ethoxy]phenyl]methyl]-1,3-thiazolidine-2,4-dione hydrochloride per ml is administered to the subject once per day.

Compound 1 or Compound 2, or pharmaceutical composition comprising Compound 1 or Compound 2 can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration to a subject. Dosage forms depend on the route administration. Dosage forms include, but are not limited to, tablets, dragees, slow release lozenges, capsules, mouth rinses and mouth washes, gels, hair rinses, hair gels, and shampoos, and suppositories, as well as suitable solutions for administration by intravenous infusion, and suitable suspensions for administration subcutaneous injection, and suitable powders for reconstitution. Parenteral administration can be accomplished using a needle and syringe or using other technique known in the art.

The pharmaceutical compositions of provided herein may be administered to any subject which may experience the beneficial effects of Compound 1 or Compound 2. Foremost among such subjects are mammals, e.g., humans, although the methods and compositions provided herein are not intended to be so limited. Other subjects include veterinary animals, e.g., cows, sheep, pigs, horses, dogs, cats and the like. In one embodiment, the subject is a human. In another embodiment, the subject is a human infected with a human coronavirus.

The pharmaceutical preparations provided herein are manufactured by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries can be suitable flow-regulating agents and lubricants. Suitable auxiliaries include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of Compound 1 or Compound 2 may be administered to a subject. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension including, for example, sodium carboxymethyl cellulose, sorbitol, and/or dextran. Optionally, the suspension may also contain stabilizers and other additives.

Therapeutically effective amounts of Compound 1 or Compound 2 formulated in accordance with standard pharmaceutical practices are administered to a subject in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

Pharmaceutical compositions include those wherein Compound 1 or Compound 2 is administered in an effective amount to achieve its intended purpose. The exact formulation, route of administration, and dosage is determined by an individual physician in view of the diagnosed condition or disease. Dosage amount and interval can be adjusted individually to provide levels of Compound 1 or Compound 2 that is sufficient to maintain therapeutic effects.

Toxicity and therapeutic efficacy of Compound 1 or Compound 2 can be determined by standard pharmaceutical procedures in cell cultures or experimental animals, e.g., for determining the maximum tolerated dose (MTD) of a compound, which defines as the highest dose that causes no toxicity in a subject. The dose ratio between the maximum tolerated dose and therapeutic effects (e.g., reduction of inflammatory response, e.g., reduction of cytokines) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

The therapeutically effective amount of Compound 1 or Compound 2 required for use in therapy varies with the nature of the disease being treated, the length of time that activity is desired, and the age and the condition of the subject, and ultimately is determined by the attendant physician. For example, dosage amounts and intervals can be adjusted individually to provide plasma levels of Compound 1 that are sufficient to maintain the desired therapeutic effects. The desired dose conveniently can be administered in a single dose, or as multiple doses administered at appropriate intervals, for example as one, two, three, four or more subdoses per day.

### III. Kits

In another embodiment, the present disclosure provides kits comprising Compound 1 or Compound 2 (or a composition comprising Compound 1 or Compound 2) packaged in a manner that facilitates its use to practice methods of the present disclosure. Compound 1 or Compound 2 may be provided in any suitable dosage for administration to a subject, e.g., as an oral suspension.

In one embodiment, the kit includes Compound 1 or Compound 2, or a composition thereof, packaged in a container, such as a sealed bottle or vessel, with a label affixed to the container or included in the kit that describes use of the compound or composition to practice the methods of the disclosure. In one embodiment, Compound 1 or Compound 2, or a composition thereof, packaged in a unit dosage form. The kit may include a single dose or multiple doses of Compound 1 or Compound 2, or a pharmaceutical composition thereof.

In another embodiment, the kit further includes a second container comprising a pharmaceutical excipient for dilution or suspension of Compound 1 or Compound 2, or pharmaceutical composition thereof. In some embodiments, Compound 1 or Compound 2, or pharmaceutical composition thereof, provided in the first container and the pharmaceutical excipient for dilution or suspension provided in second container are combined to form one unit dosage form.

In another embodiment, the kit further includes a device or instrument for assisting with the administration of Compound 1 or Compound 2 according to the intended route of administration to the subject. Such a device may be a syringe, catheter, or any such medically approved delivery means.

### IV. Definitions

The terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language, e.g., “such as,” provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

As used herein, the terms “treat,” “treating,” “treatment,” and the like refer to eliminating, reducing, or ameliorating an inflammatory lung condition or disease caused by a viral infection, and/or symptoms associated therewith. Symptoms of the inflammatory lung condition or disease caused by a viral infection include, but are not limited to, fever, cough, and shortness of breath. Although not precluded, treating an inflammatory lung condition or disease caused by a viral infection does not require that the condition, disease, or symptoms associated therewith be completely eliminated. However, in one embodiment, administration of Compound 1 or Compound 2 leads to complete elimination of the inflammatory lung condition or disease, and the associated symptoms.

The term “therapeutically effective amount,” as used herein, refers to that amount of Compound 1 or Compound 2 and/or one or more optional therapeutic agents sufficient to result in amelioration of one or more symptoms of the inflammatory lung condition or disease, or prevent advancement of the inflammatory lung condition or disease, or cause regression of the inflammatory lung condition or disease caused by a viral infection. For example, with respect to the treatment of an inflammatory lung condition or disease caused by a viral infection, e.g., a human coronavirus infection, in one embodiment, a therapeutically effective amount will refer to the amount of Compound 1 or Compound 2 that causes a therapeutic response, e.g., decrease inflammation and/or inflammatory response in the subject by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more. With respect to decreasing the level of one or more cytokines in a subject, in one embodiment, a therapeutically effective amount will refer to the amount of Compound 1 or Compound 2 that causes an overall reduction of about 1% to about 300% in the level of the cytokine(s) in the subject or a biological sample taken from the subject. Cytokine expression levels in a subject can be determined using assay techniques known in the art. See, e.g., Amsen et al., Methods Mol Biol. 511: 107-142 (2009). Exemplary non-limiting assays include PCR based assays, in situ hybridisation assays, flow cytometry assays, and immunological or immunohistochemical assays.

The term “container” means any receptacle and closure therefore suitable for storing, shipping, dispensing, and/or handling a Compound 1 or Compound 2. Non-limiting exemplary containers include vials, ampules, bottles, and syringes.

The term “insert” means information accompanying a pharmaceutical product that provides a description of how to administer the product, along with the safety and efficacy data required to allow the physician, pharmacist, and subject to make an informed decision regarding use of the product. The package insert generally is regarded as the “label” for a pharmaceutical product.

In some embodiments, when administered in combination, two or more therapeutic agents can have a synergistic effect. The terms “synergy,” “synergistic,” “synergistically” and derivations thereof, such as in a “synergistic effect” or a “synergistic combination” or a “synergistic composition” as used herein refer to circumstances under which the biological activity of a combination of an agent and at least one additional therapeutic agent is greater than the sum of the biological activities of the respective agents when administered individually. For example, the term “synergistically effective” as used herein refers to the interaction between Compound 1 or Compound 2 and another therapeutic agent(s) that causes the total effect of the drugs to be greater than the sum of the individual effects of each drug. Berenbaum, Pharmacological Reviews 41:93-141 (1989).

Synergy can be expressed in terms of a “Synergy Index (SI),” which generally can be determined by the method described by F. C. Kull et al. Applied Microbiology 9, 538 (1961), from the ratio determined by:

QₐQ_{A}+Q_{b}Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

The terms “intermittent dose administration,” “intermittent dosing schedule,” and similar terms as used herein refer to non-continuous administration of a Compound 1 or Compound 2 to a subject. For example administration of Compound 1 or Compound 2 to a subject on Monday, Wednesday, and Friday and no administration on Tuesday, Thursday, Saturday, and Sunday is a non-limiting exemplary intermittent dosing schedule.

Intermittent dose administration of Compound 1 or Compound 2 may maintain or improve the efficacy achieved with continuous dosing, but with less side-effects, e.g., less body weight loss. Intermittent dose administration regimens useful in the present disclosure encompass any discontinuous administration regimen that provides a therapeutically effective amount of a Compound 1 or Compound 2 to a subject in need thereof. Intermittent dosing regimens can use equivalent, lower, or higher doses of Compound 1 or Compound 2 than would be used in continuous dosing regimens. Advantages of intermittent dose administration of a Compound 1 or Compound 2 include, but are not limited to, improved safety, decreased toxicity, e.g., decreased weight loss, increased exposure, increased efficacy, and/or increased subject compliance. These advantages may be realized when Compound 1 or Compound 2 is administered as a single agent or when administered in combination with one or more optional therapeutic agents. On the day Compound 1 or Compound 2 is scheduled to be administered to the subject, administration can occur in a single or in divided doses, e.g., once-a-day, twice-a-day, three times a day, four times a day or more. Dosing can also occur via any suitable route, e.g., orally, intravenously, or subcutaneously. In one embodiment, Compound 1 or Compound 2 is administered to the subject orally. In another embodiment, Compound 1 or Compound 2 is administered to the subject once (QD) or twice (BID) on the day the compound is scheduled to be administered.

The phrase “in combination” as used in connection with the administration of Compound 1 or Compound 2 and one or more optional therapeutic agents to a subject means that Compound 1 or Compound 2 and the one or more optional therapeutic agents can be administered to the subject together, e.g., as part of a single pharmaceutical composition or formulation, or separately, e.g., as part of two or more separate pharmaceutical compositions or formulations. The phrase “in combination” as used in connection with the administration of Compound 1 or Compound 2 and the one or more optional therapeutic agents to a subject is thus intended to embrace administration of Compound 1 or Compound 2 and the one or more optional therapeutic agents in a sequential manner, wherein Compound 1 or Compound 2 and the one or more optional therapeutic agents are administered to the subject at a different time, as well as administration concurrently, or in a substantially simultaneous manner. Sequential or substantially simultaneous administration of Compound 1 or Compound 2 and the one or more optional therapeutic agents can be accomplished by any appropriate route including, but not limited to, oral routes, intravenous routes, subcutaneous routes, intramuscular routes, etc. Compound 1 or Compound 2 and the one or more optional therapeutic agents can be administered by the same route or by different routes. For example, the one or more optional therapeutic agents of the combination may be administered by intravenous injection while Compound 1 of the combination may be administered orally. Compound 1 or Compound 2 and the one or more optional therapeutic agents may also be administered in alternation. In one embodiment, Compound 1 or Compound 2 and the one or more optional therapeutic agents are administered to a subject separately, e.g., as part of two or more separate pharmaceutical compositions or formulations.

The term “human coronavirus” as used herein refers to a positive-stranded RNA virus that has a lipid envelope studded with club-shaped projections that infect humans, and mutated strains thereof. Sexton et al., Journal of Virology 90:7415-7428 (2016).

The terms “HCoV-229E,” “HCoV-OC43,” “HCoV-NL63,” “HCoV-HKU1,” “SARS-CoV,” “MERS-CoV,” and “SARS-CoV-2” as used herein refer to the coronavirus pathogens known to infect humans. Lim et al., Diseases 2016, 4, 26; doi:10.3390/diseases4030026; Lai et al., International Journal of Antimicrobial Agent 55:1-9 (2020).

The terms “acute respiratory distress syndrome” or “ARDS” as used herein refers to a lung condition that, inter alia, leads to low oxygen levels in the blood. Raniei et al., JAMA 307:2526-2533 (2012).

The terms “systemic inflammatory response syndrome” or “SIRS” as used herein refers to an inflammatory state affecting the entire body. Jaffer et al., HSR Proc Intensive Care Cardiovasc Anesth. 2: 161-175 (2010). Complications of SIRS include, but are not limited to, acute kidney injury, shock, and multiple organ dysfunction syndrome. Manifestations of SIRS include, but are not limited to, body temperature less than 36° C. (96.8° F.) or greater than 38° C. (100.4° F.); heart rate greater than 90 beats per minute; tachypnea (high respiratory rate), with greater than 20 breaths per minute; or, an arterial partial pressure of carbon dioxide less than 4.3 kPa (32 mm Hg); and white blood cell count less than 4000 cells/mm³ (4×109 cells/L) or greater than 12,000 cells/mm³ (12×109 cells/L). When two or more of these criteria are met with or without evidence of infection, patients may be diagnosed with SIRS. Bone et al., Chest 101:1644-1655 (1992).

The term “Coronavirus Disease 2019” or “COVID-19” as used herein refers to the viral respiratory disease caused by SARS-CoV-2.

The terms “level,” “level of expression,” “expression level,” and the like are used interchangeably and refer to the amount of cytokine present in a subject or biological sample taken from the subject.

The term “decreased level” and the like as used herein refers to an overall reduction of about 1% to about 1000%, e.g., about 1% to about 300%, about 1% to about 100%, about 5% to about 75%, or about 30% to about 70%, in the level of a cytokine in the subject or a biological sample taken from the subject as compared to a reference value or another biological sample from a subject. In one embodiment, a decreased level refers to an overall reduction of about 5%, about 10%, about 15%, about 20%, about 25%, about 30%, about 35%, about 40%, about 45%, about 50%, about 55%, about 60% about 65%, about 70%, about 75%, about 80%, about 85%, about 90%, about 95%, about 100%, about 125%, about 150%, about 175%, about 200%, about 225%, about 2 50%, about 275%, about 300%, or about 500% in the level of a cytokine in the subject or a biological sample taken from the subject as compared to a reference value or another biological sample from a subject.

The term “increased level” and the like as used herein refers to an overall increase of about 1% to about 1000%, e.g., about 1% to about 300%, about 1% to about 100%, about 5% to about 50%, or about 30% to about 70%, in the level of a cytokine in the subject or a biological sample taken from the subject as compared to a reference value or another biological sample from a subject. In one embodiment, an increased level refers to an overall increase of about 5%, about 10%, about 15%, about 20%, about 25%, about 30%, about 35%, about 40%, about 45%, about 50%, about 55%, about 60% about 65%, about 70%, about 75%, about 80%, about 85%, about 90%, about 95%, about 100%, about 125%, about 150%, about 175%, about 200%, about 225%, about 2 50%, about 275%, about 300%, or about 500% in the level of a cytokine in the subject or a biological sample taken from the subject as compared to a reference value or another biological sample from a subject.

The term “reference value” as used herein refers to a predetermined value of the amount of a cytokine present in a subject not having an inflammatory lung condition or disease caused by a viral infection, acute inflammation of the lung, or interstitial lung disease or biological sample taken from a subject not having an inflammatory lung condition or disease caused by a viral infection, acute inflammation of the lung, or interstitial lung disease. As the skilled artisan will appreciate, the reference value is predetermined and set to meet the requirements in terms of, for example, specificity and/or sensitivity of the particular cytokine detection assay used to quantify the cytokine. It may be, for example, that assay sensitivity or specificity, respectively, has to be set to certain limits, e.g., 80%, 90%, or 95%. These requirements may also be defined in terms of positive or negative predictive values. Nonetheless, based on the teaching of the present disclosure it will always be possible to arrive at the reference value meeting those requirements. In one embodiment, the reference value is determined in healthy individuals. The reference value, in one embodiment, has been predetermined in the disease entity to which the subject belongs, e.g., an inflammatory lung condition or disease caused by a viral infection. In certain embodiments, the reference value can be set to any percentage between, e.g., 25% and 75% of the overall distribution of the values in a disease entity investigated. In other embodiments, the reference level can be set to, for example, the median, tertiles, quartiles, or quintiles as determined from the overall distribution of the values in a disease entity investigated or in a given population. In one embodiment, the reference value is set to the median value as determined from the overall distribution of the values in a disease entity investigated. In one embodiment, the reference value may depend on the gender of the patient, e.g., males and females may have different reference levels, and/or the severity of the condition or disease of the subject.

Cytokine expression levels may be determined by one of a number of known in vitro assay techniques, such as PCR based assays, in situ hybridisation assays, flow cytometry assays, immunological or immunohistochemical assays. In one embodiment, cytokine expression levels are determined by quantitative PCR, proteomics, immunological methods, proteomics, e.g., ELISA, enzyme-linked immunosorbent spot (ELIspot), antibody array assays and bead-based assays, flow cytometry, or a microfluidic platform. In another embodiment, cytokine expression levels are determined by an immunological assay, e.g., ELISA.

By way of example using IL-4 as a non-limiting, exemplary cytokine, suitable techniques involve a method of detecting the level of IL-4 or a receptor for IL-4 in a sample by contacting the biological sample with an agent capable of binding IL-4 or a receptor for IL-4 and detecting the formation of a complex of the agent and IL-4 or receptor for IL-4. The agent may be any suitable binding molecule, e.g. an antibody, polypeptide, peptide, oligonucleotide, aptamer or small molecule, and may optionally be labelled to permit detection, e.g. visualization, of the complexes formed. Suitable labels and means for their detection are well known to those in the art and include fluorescent labels (e.g. fluorescein, rhodamine, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, Cy5), isotope markers, radioisotopes (e.g. 32P, 33P, 35S), chemiluminescence labels (e.g. acridinium ester, luminol, isoluminol), enzymes (e.g. peroxidase, alkaline phosphatase, glucose oxidase, beta-galactosidase, luciferase), antibodies, ligands and receptors. Detection techniques are well known to those of skill in the art and can be selected to correspond with the labelling agent. Suitable techniques include PCR amplification of oligonucleotide tags, mass spectrometry, detection of fluorescence or color, e.g. upon enzymatic conversion of a substrate by a reporter protein, or detection of radioactivity. Assays may be configured to quantify the amount of IL-4 or receptor for IL-4 in a sample. Quantified amounts of IL-4 or receptor for TL-4 from a test sample from a subject may be compared with reference values or another sample from the subject, and the comparison can be used to determine whether the test sample contains an amount of IL-4 or receptor for IL-4 that is higher or lower than that of the reference value or the other sample to a selected degree of statistical significance. In one embodiment, for example, the amount of IL-4 in a biological sample taken from the subject before administration of Compound 1 or Compound 2 may be compared against the amount of IL-4 in a biological sample taken from the subject after administration of Compound 1 or Compound 2, e.g., after 1 day, after 2 days, after 3 days, after 4 days, after 5 days, after 6 days, after 1 week, after 2 weeks, after 3 weeks, or after 4 weeks of administration.

A biological sample obtained from a subject may be of any kind. The sample may be taken from any tissue or bodily fluid, e.g. a blood sample, blood-derived sample, serum sample, lymph sample, semen sample, saliva sample, synovial fluid sample. A blood-derived sample may be a selected fraction of a subject's blood, e.g. a selected cell-containing fraction or a plasma or serum fraction. A sample may comprise a tissue sample or biopsy; or cells isolated from a subject. Samples may be collected by known techniques, such as biopsy or needle aspirate. Biological samples may be stored and/or processed for subsequent determination of cytokine expression levels.

In some embodiments a biological sample is a lung tissue sample. In other embodiments, a biological sample is bronchoalveolar lavage fluid.

The term “IP-10” as used herein refers to the cytokine encoded by the CXCL10 gene. IP-10 is also known as C-X-C motif chemokine ligand 10 (CXCL10) or small-inducible cytokine B10.

The term “IL-4” as used herein refers to the cytokine known as interleukin 4.

The term “IFNα2” as used herein refers to the cytokine encoded by the IFNA2 gene. IFNα2 is also known as interferon alpha-2.

The term “IFNγ” as used herein refers to the cytokine encoded by the IFNG gene. IFNγ is also known as type II interferon or interferon gamma.

The term “TNFα” as used herein refers to the cytokine known as tumor necrosis factor alpha or TNF-alpha.

The term “MCP1” as used herein refers to the cytokine also known as monocyte chemoattractant protein 1, chemokine (C-C motif) ligand 2 (CCL2), or small inducible cytokine A2.

The term “IL-6” as used herein refers to the cytokine encoded by the IL6 gene. IL-6 is also known as interleukin 6.

The term “MIP1-α” as used herein refers to the cytokine encoded by the CCL gene. MIP1-α is also known as macrophage inflammatory protein 1-alpha or chemokine (C-C motif) ligand 3 (CCL3).

The term “IL-12p40” as used herein refers to the cytokine encoded by the IL12B gene. IL-12p40 is also known as IL-12B, natural killer cell stimulatory factor 2, cytotoxic lymphocyte maturation factor p40, or interleukin-12 subunit p40.

The term “CXCL 9” as used herein refers to the cytokine as known as monokine induced by gamma interferon (MIG).

The term “RANTES” as used herein refers to the cytokine encoded by the CCL5 gene.

The term “CD45” as used herein refers to the transmembrane protein tyrosine phosphatase also known as leukocyte common antigen, a pan leukocyte marker.

### V. Particular Embodiments

The disclosure provides the following particular embodiments with respect decreasing the level of IP-10, IL-4, IFNα2, IFNγ, TNFα, MCP1, IL-6, MIP1-α, IL-12p40, CXCL 9, RANTES, CD45⁺ cells, T cells, or neutrophils, or a combination thereof, in a subject in need thereof.

Embodiment 1. A method for decreasing the level of IP-10, IL-4, IFNα2, IFNγ, TNFα, MCP1, IL-6, or MIP1-α, IL-12p40, CXCL 9, RANTES, CD45⁺ cells, T cells, or neutrophils, or a combination thereof, in a subject in need thereof, the method comprising administering to the subject a therapeutically effective amount of Compound 1 or Compound 2, wherein the subject has an inflammatory lung condition or disease caused by a viral infection, acute inflammation of the lung, or interstitial lung disease.

Embodiment 2. The method of Embodiment 1, wherein the subject has an inflammatory lung condition or disease caused by a viral infection.

Embodiment 3. The method of Embodiment 2, wherein the viral infection is a human coronavirus infection, an influenza virus infection, or a HIV virus infection.

Embodiment 4. The method of Embodiment 3, wherein the human coronavirus is HCoV-229E, HCoV-OC43, HCoV-NL63, HCoV-HKU1, SARS-CoV, MERS-CoV, or SARS-CoV-2, or a mutated strain thereof.

Embodiment 5. The method of Embodiment 4, wherein the human coronavirus is SARS-CoV-2.

Embodiment 6. The method of Embodiment 5, wherein the human coronavirus is a mutated strain of SARS-CoV-2.

Embodiment 7. The method of any one of Embodiments 2-6, wherein the inflammatory lung condition or disease caused by the viral infection is hypercytokinaemia, haemophagocytic lymphohistiocytosis, pneumonia, acute respiratory distress syndrome, or systemic inflammatory response syndrome.

Embodiment 8. The method of Embodiment 7, wherein the inflammatory lung condition or disease caused by the viral infection is acute respiratory distress syndrome.

Embodiment 9. The method of Embodiment 1, wherein the subject has acute inflammation of the lung.

Embodiment 10. The method of Embodiment 9, wherein the acute inflammation of the lung is pneumonia or acute respiratory distress syndrome.

Embodiment 11. The method of Embodiment 1, wherein the subject has an interstitial lung disease.

Embodiment 12. The method of Embodiment 11, wherein the interstitial lung disease is idiopathic pulmonary fibrosis.

Embodiment 13. The method of any one of Embodiments 1-12, further comprising administering one or more optional therapeutic agents to the subject.

Embodiment 14. The method of any one of Embodiments 1-13, comprising administering the hydrochloride salt of racemic 5-[[4-[2-[5-(1-hydroxyethyl)pyridin-2-yl]ethoxy]phenyl]methyl]-1,3-thiazolidine-2,4-dione to the subject.

Embodiment 15. The method of Embodiment 14, wherein the hydrochloride salt of racemic 5-[[4-[2-[5-(1-hydroxyethyl)pyridin-2-yl]ethoxy]phenyl]methyl]-1,3-thiazolidine-2,4-dione is administered to the subject as an oral suspension.

Embodiment 16. The method of Embodiment 15, wherein about 5 mL to about 25 mL of an oral suspension comprising about 15 mg of the hydrochloride salt of racemic 5-[[4-[2-[5-(1-hydroxyethyl)pyridin-2-yl]ethoxy]phenyl]methyl]-1,3-thiazolidine-2,4-dione per mL is administered to the subject once per day.

Embodiment 17. The method of any one of Embodiments 1-16, wherein the level of IP-10 is decreased.

Embodiment 18. The method of any one of Embodiments 1-17, wherein the level of IL-4 is decreased.

Embodiment 19. The method of any one of Embodiments 1-18, wherein the level of IFNα2 is decreased.

Embodiment 20. The method of any one of Embodiments 1-19, wherein the level of IFNγ is decreased.

Embodiment 21. The method of any one of Embodiments 1-20, wherein the level of TNFα is decreased.

Embodiment 22. The method of any one of Embodiments 1-21, wherein the level of MCP1 is decreased.

Embodiment 23. The method of any one of Embodiments 1-22, wherein the level of IL-6 is decreased.

Embodiment 24. The method of any one of Embodiments 1-23, wherein the level of MIP1-α is decreased.

Embodiment 25. The method of any one of Embodiments 1-24, wherein the level of IP-10, IL-4, IFNα2, IFNγ, TNFα, MCP1, IL-6, or MIP1-α, or a combination thereof, is measured by immunological methods, e.g., ELISA.

Embodiment 26. The method of any one of Embodiments 1-25, wherein the level of IP-10, IL-4, IFNα2, IFNγ, TNFα, MCP1, IL-6, or MIP1-α, or a combination thereof, is decreased by about 1% to about 100%, e.g., about 5% to about 95%, e.g., about 10% to about 90%, e.g., about 20% to about 85%, e.g., about 30% to about 70%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

Embodiment 27. The method of Embodiment 26, wherein the level of IP-10 is decreased by about 20% to about 85%.

Embodiment 28. The method of Embodiments 26 or 27, wherein the level of IL-4 is decreased by about 20% to about 85%.

Embodiment 29. The method of any one of Embodiments 26-28, wherein the level of IFNα2 is decreased by about 20% to about 85%.

Embodiment 30. The method of any one of Embodiments 26-29, wherein the level of IFNγ is decreased by about 20% to about 85%.

Embodiment 31. A composition comprising (i) Compound 1 and a pharmaceutically acceptable carrier; or (ii) Compound 2 and a pharmaceutically acceptable carrier, for use in decreasing the level of IP-10, IL-4, IFNα2, IFNγ, TNFα, MCP1, IL-6, MIP1-α, IL-12p40, CXCL 9, RANTES, CD45⁺ cells, neutrophils, or T cells, or a combination thereof, in a subject, wherein the subject has inflammatory lung condition or disease caused by a viral infection, acute inflammation of the lung, or interstitial lung disease.

Embodiment 32. The composition of Embodiment 31, wherein the subject has an inflammatory lung condition or disease caused by a viral infection.

Embodiment 33. The composition of Embodiment 32, wherein the viral infection is a human coronavirus infection, an influenza virus infection, or a HIV virus infection.

Embodiment 34. The composition of Embodiment 33, wherein the human coronavirus is HCoV-229E, HCoV-OC43, HCoV-NL63, HCoV-HKU1, SARS-CoV, MERS-CoV, or SARS-CoV-2, or a mutated strain thereof.

Embodiment 35. The composition of Embodiment 34, wherein the human coronavirus is SARS-CoV-2.

Embodiment 36. The composition of Embodiment 35, wherein the human coronavirus is a mutated strain of SARS-CoV-2.

Embodiment 37. The composition of any one of Embodiments 32-36, wherein the inflammatory lung condition or disease caused by the viral infection is hypercytokinaemia, haemophagocytic lymphohistiocytosis, pneumonia, acute respiratory distress syndrome, or systemic inflammatory response syndrome.

Embodiment 38. The composition of Embodiment 37, wherein the inflammatory lung condition or disease caused by the viral infection is acute respiratory distress syndrome.

Embodiment 39. The composition of Embodiment 31, wherein the subject has acute inflammation of the lung.

Embodiment 40. The composition of Embodiment 39, wherein the acute inflammation of the lung is pneumonia or acute respiratory distress syndrome.

Embodiment 41. The composition of Embodiment 31, wherein the subject has an interstitial lung disease.

Embodiment 42. The composition of Embodiment 41, wherein the interstitial lung disease is idiopathic pulmonary fibrosis.

Embodiment 43. The composition of any one of Embodiments 31-42, wherein the composition is to be administered with one or more optional therapeutic agents to the subject.

Embodiment 44. The composition of any one of Embodiments 31-43, comprising administering the hydrochloride salt of racemic 5-[[4-[2-[5-(1-hydroxyethyl)pyridin-2-yl]ethoxy]phenyl]methyl]-1,3-thiazolidine-2,4-dione to the subject.

Embodiment 45. The composition of Embodiment 44, wherein the hydrochloride salt of racemic 5-[[4-[2-[5-(1-hydroxyethyl)pyridin-2-yl]ethoxy]phenyl]methyl]-1,3-thiazolidine-2,4-dione is administered to the subject as an oral suspension.

Embodiment 46. The composition Embodiment 45, wherein about 5 mL to about 25 mL of an oral suspension comprising about 15 mg of the hydrochloride salt of racemic 5-[[4-[2-[5-(1-hydroxyethyl)pyridin-2-yl]ethoxy]phenyl]methyl]-1,3-thiazolidine-2,4-dione per mL is administered to the subject once per day.

Embodiment 47. The composition of any one of Embodiments 31-46, wherein the level of IP-10 is decreased.

Embodiment 48. The composition of any one of Embodiments 31-47, wherein the level of IL-4 is decreased.

Embodiment 49. The composition of any one of Embodiments 31-48, wherein the level of IFNα2 is decreased.

Embodiment 50. The composition of any one of Embodiments 31-49, wherein the level of IFNγ is decreased.

Embodiment 51. The composition of any one of Embodiments 31-50, wherein the level of TNFα is decreased.

Embodiment 52. The composition of any one of Embodiments 31-51, wherein the level of MCP1 is decreased.

Embodiment 53. The composition of any one of Embodiments 31-52, wherein the level of IL-6 is decreased.

Embodiment 54. The composition of any one of Embodiments 31-53, wherein the level of MIP1-α is decreased.

Embodiment 55. The composition of any one of Embodiments 31-44, wherein the level of IP-10, IL-4, IFNα2, IFNγ, TNFα, MCP1, IL-6, or MIP1-α, or a combination thereof, is measured by quantitative PCR, proteomics, immunological methods, proteomics, e.g., ELISA, enzyme-linked immunosorbent spot (ELIspot), antibody array assays and bead-based assays, flow cytometry, or a microfluidic platform.

Embodiment 56. The composition of any one of Embodiments 31-55, wherein the level of IP-10, IL-4, IFNα2, IFNγ, TNFα, MCP1, IL-6, or MIP1-α, or a combination thereof, is decreased about 1% to about 100%.

Embodiment 57. The composition of Embodiment 56, wherein the level of IP-10 is decreased by about 20% to about 85%.

Embodiment 58. The composition of Embodiments 56 or 57, wherein the level of IL-4 is decreased by about 20% to about 85%.

Embodiment 59. The composition of any one of Embodiments 56-58, wherein the level of IFNα2 is decreased by about 20% to about 85%.

Embodiment 60. The composition of any one of Embodiments 56-59, wherein the level of IFNγ is decreased by about 20% to about 85%.

Embodiment 61. Compound 1 or Compound 2 for use in decreasing the level of IP-10, IL-4, IFNα2, IFNγ, TNFα, MCP1, IL-6, MIP1-α, IL-12p40, CXCL 9, RANTES, CD45⁺ cells, neutrophils, or T cells, or a combination thereof, in a subject, wherein the subject has inflammatory lung condition or disease caused by a viral infection, acute inflammation of the lung, or interstitial lung disease.

Embodiment 62. The Compound 1 or Compound 2 for use of Embodiment 61, wherein the subject has an inflammatory lung condition or disease caused by a viral infection.

Embodiment 63. The Compound 1 or Compound 2 for use of Embodiment 62, wherein the viral infection is a human coronavirus infection, an influenza virus infection, or a HIV virus infection.

Embodiment 64. The Compound 1 or Compound 2 for use of Embodiment 63, wherein the human coronavirus is HCoV-229E, HCoV-OC43, HCoV-NL63, HCoV-HKU1, SARS-CoV, MERS-CoV, or SARS-CoV-2, or a mutated strain thereof.

Embodiment 65. The Compound 1 or Compound 2 for use of Embodiment 64, wherein the human coronavirus is SARS-CoV-2.

Embodiment 66. The Compound 1 or Compound 2 for use of Embodiment 65, wherein the human coronavirus is a mutated strain of SARS-CoV-2.

Embodiment 67. The Compound 1 or Compound 2 for use of any one of Embodiments 62-66, wherein the inflammatory lung condition or disease caused by the viral infection is hypercytokinaemia, haemophagocytic lymphohistiocytosis, pneumonia, acute respiratory distress syndrome, or systemic inflammatory response syndrome.

Embodiment 68. The Compound 1 or Compound 2 for use of Embodiment 67, wherein the inflammatory lung condition or disease caused by the viral infection is acute respiratory distress syndrome.

Embodiment 69. The Compound 1 or Compound 2 for use of Embodiment 61, wherein the subject has acute inflammation of the lung.

Embodiment 70. The Compound 1 or Compound 2 for use of Embodiment 69, wherein the acute inflammation of the lung is pneumonia or acute respiratory distress syndrome.

Embodiment 71. The Compound 1 or Compound 2 for use of Embodiment 61, wherein the subject has an interstitial lung disease.

Embodiment 72. The Compound 1 or Compound 2 for use of Embodiment 71, wherein the interstitial lung disease is idiopathic pulmonary fibrosis.

Embodiment 73. The Compound 1 or Compound 2 for use of any one of Embodiments 61-72 further comprising administering one or more optional therapeutic agents to the subject.

Embodiment 74. The Compound 1 or Compound 2 for use of any one of Embodiments 61-73, comprising administering the hydrochloride salt of racemic 5-[[4-[2-[5-(1-hydroxyethyl)pyridin-2-yl]ethoxy]phenyl]methyl]-1,3-thiazolidine-2,4-dione to the subject.

Embodiment 75. The Compound 1 or Compound 2 for use of Embodiment 74, wherein the hydrochloride salt of racemic 5-[[4-[2-[5-(1-hydroxyethyl)pyridin-2-yl]ethoxy]phenyl]methyl]-1,3-thiazolidine-2,4-dione is to be administered to the subject as an oral suspension.

Embodiment 76. The Compound 1 or Compound 2 for use of Embodiment 75, wherein about 5 mL to about 25 mL of an oral suspension comprising about 15 mg of the hydrochloride salt of racemic 5-[[4-[2-[5-(1-hydroxyethyl)pyridin-2-yl]ethoxy]phenyl]methyl]-1,3-thiazolidine-2,4-dione per mL is administered to the subject once per day.

Embodiment 77. The Compound 1 or Compound 2 for use of any one of Embodiments 61-76, wherein the level of IP-10 is decreased.

Embodiment 78. The Compound 1 or Compound 2 for use of any one of Embodiments 61-77, wherein the level of IL-4 is decreased.

Embodiment 79. The Compound 1 or Compound 2 for use of any one of Embodiments 61-78, wherein the level of IFNα2 is decreased.

Embodiment 80. The Compound 1 or Compound 2 for use of any one of Embodiments 61-79, wherein the level of IFNγ is decreased.

Embodiment 81. The Compound 1 or Compound 2 for use of any one of Embodiments 61-80, wherein the level of TNFα is decreased.

Embodiment 82. The Compound 1 or Compound 2 for use of any one of Embodiments 61-81, wherein the level of MCP1 is decreased.

Embodiment 83. The Compound 1 or Compound 2 for use of any one of Embodiments 61-82, wherein the level of IL-6 is decreased.

Embodiment 84. The Compound 1 or Compound 2 for use of any one of Embodiments 61-83, wherein the level of MIP1-α is decreased.

Embodiment 85. The Compound 1 or Compound 2 for use of any one of Embodiments 61-84, wherein the level of IP-10, IL-4, IFNα2, IFNγ, TNFα, MCP1, IL-6, or MIP1-α, or a combination thereof, is measured by immunological methods, e.g., ELISA.

Embodiment 86. The Compound 1 or Compound 2 for use of any one of Embodiments 61-85, wherein the level of IP-10, IL-4, IFNα2, IFNγ, TNFα, MCP1, IL-6, or MIP1-α, or a combination thereof, is decreased by about 1% to about 100%, e.g., about 5% to about 95%, e.g., about 10% to about 90%, e.g., about 20% to about 85%, e.g., about 30% to about 70%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

Embodiment 87. The Compound 1 or Compound 2 for use of Embodiment 86, wherein the level of IP-10 is decreased by about 20% to about 85%.

Embodiment 88. The Compound 1 or Compound 2 for use of Embodiments 86 or 87, wherein the level of IL-4 is decreased by about 20% to about 85%.

Embodiment 89. The Compound 1 or Compound 2 for use of any one of Embodiments 86-88, wherein the level of IFNα2 is decreased by about 20% to about 85%.

Embodiment 90. The Compound 1 or Compound 2 for use of any one of Embodiments 86-89, wherein the level of IFNγ is decreased by about 20% to about 85%.

Embodiment 91. Use of Compound 1 or Compound 2 in the manufacture of a medicament for decreasing the level of IP-10, IL-4, IFNα2, IFNγ, TNFα, MCP1, IL-6, MIP1-α, IL-12p40, CXCL 9, RANTES, CD45⁺ cells, T cells, or neutrophils, or a combination thereof, in a subject, wherein the subject has inflammatory lung condition or disease caused by a viral infection, acute inflammation of the lung, or interstitial lung disease.

Embodiment 92. The use of Embodiment 91, wherein the subject has an inflammatory lung condition or disease caused by a viral infection.

Embodiment 93. The use of Embodiment 92, wherein the viral infection is a human coronavirus infection, an influenza virus infection, or a HIV virus infection.

Embodiment 94. The use of Embodiment 93, wherein the human coronavirus is HCoV-229E, HCoV-OC43, HCoV-NL63, HCoV-HKU1, SARS-CoV, MERS-CoV, or SARS-CoV-2, or a mutated strain thereof.

Embodiment 95. The use of Embodiment 94, wherein the human coronavirus is SARS-CoV-2.

Embodiment 96. The use of Embodiment 95, wherein the human coronavirus is a mutated strain of SARS-CoV-2.

Embodiment 97. The use of any one of Embodiments 92-96, wherein the inflammatory lung condition or disease caused by the viral infection is hypercytokinaemia, haemophagocytic lymphohistiocytosis, pneumonia, acute respiratory distress syndrome, or systemic inflammatory response syndrome.

Embodiment 98. The use of Embodiment 97, wherein the inflammatory lung condition or disease caused by the viral infection is acute respiratory distress syndrome.

Embodiment 99. The use of Embodiment 91, wherein the subject has acute inflammation of the lung.

Embodiment 100. The use of Embodiment 99, wherein the acute inflammation of the lung is pneumonia or acute respiratory distress syndrome.

Embodiment 101. The use of Embodiment 91, wherein the subject has an interstitial lung disease.

Embodiment 102. The use of Embodiment 101, wherein the interstitial lung disease is idiopathic pulmonary fibrosis.

Embodiment 103. The use of any one of Embodiments 91-102 further comprising administering one or more optional therapeutic agents to the subject.

Embodiment 104. The use of any one of Embodiments 91-103, comprising administering the hydrochloride salt of racemic 5-[[4-[2-[5-(1-hydroxyethyl)pyridin-2-yl]ethoxy]phenyl]methyl]-1,3-thiazolidine-2,4-dione to the subject.

Embodiment 105. The use of Embodiment 104, wherein the hydrochloride salt of racemic 5-[[4-[2-[5-(1-hydroxyethyl)pyridin-2-yl]ethoxy]phenyl]methyl]-1,3-thiazolidine-2,4-dione is to be administered to the subject as an oral suspension.

Embodiment 106. The use of Embodiment 105, wherein about 5 mL to about 25 mL of an oral suspension comprising about 15 mg of the hydrochloride salt of racemic 5-[[4-[2-[5-(1-hydroxyethyl)pyridin-2-yl]ethoxy]phenyl]methyl]-1,3-thiazolidine-2,4-dione per mL is administered to the subject once per day.

Embodiment 107. The use of any one of Embodiments 91-106, wherein the level of IP-10 is decreased.

Embodiment 108. The use of any one of Embodiments 91-107, wherein the level of IL-4 is decreased.

Embodiment 109. The use of any one of Embodiments 91-108, wherein the level of IFNα2 is decreased.

Embodiment 110. The use of any one of Embodiments 91-109, wherein the level of IFNγ is decreased.

Embodiment 111. The use of any one of Embodiments 91-110, wherein the level of TNFα is decreased.

Embodiment 112. The use of any one of Embodiments 91-111, wherein the level of MCP1 is decreased.

Embodiment 113. The use of any one of Embodiments 91-112, wherein the level of IL-6 is decreased.

Embodiment 114. The use of any one of Embodiments 91-113, wherein the level of MIP1-α is decreased.

Embodiment 115. The use of any one of Embodiments 91-114, wherein the level of IP-10, IL-4, IFNα2, IFNγ, TNFα, MCP1, IL-6, or MIP1-α, or a combination thereof, is measured by immunological methods, e.g., ELISA.

Embodiment 116. The use of any one of Embodiments 91-115, wherein the level of IP-10, IL-4, IFNα2, IFNγ, TNFα, MCP1, IL-6, or MIP1-α, or a combination thereof, is decreased by about 1% to about 100%, e.g., about 5% to about 95%, e.g., about 10% to about 90%, e.g., about 20% to about 85%, e.g., about 30% to about 70%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

Embodiment 117. The use of Embodiment 116, wherein the level of IP-10 is decreased by about 20% to about 85%.

Embodiment 118. The use of Embodiments 116 or 117, wherein the level of IL-4 is decreased by about 20% to about 85%.

Embodiment 119. The use of any one of Embodiments 116-118, wherein the level of IFNα2 is decreased by about 20% to about 85%.

Embodiment 120. The use of any one of Embodiments 116-119, wherein the level of IFNγ is decreased by about 20% to about 85%.

Embodiment 121. A kit comprising Compound 1 or Compound 2 in a container and instructions for administering Compound 1 or Compound 2 to a subject having an increased level of IP-10, IL-4, IFNα2, IFNγ, TNFα, MCP1, IL-6, MIP1-α, IL-12p40, CXCL 9, RANTES, CD45⁺ cells, neutrophils, or T cells, or a combination thereof, wherein the subject has an inflammatory lung condition or disease caused by a viral infection, acute inflammation of the lung, or idiopathic pulmonary fibrosis.

Embodiment 122. The kit of Embodiment 121, wherein the subject has an inflammatory lung condition or disease caused by a viral infection.

Embodiment 123. The kit of Embodiment 122, wherein the viral infection is a human coronavirus infection, an influenza virus infection, or a HIV virus infection.

Embodiment 124. The kit of Embodiment 123, wherein the human coronavirus is HCoV-229E, HCoV-OC43, HCoV-NL63, HCoV-HKU1, SARS-CoV, MERS-CoV, or SARS-CoV-2, or a mutated strain thereof.

Embodiment 125. The kit of Embodiment 124, wherein the human coronavirus is SARS-CoV-2.

Embodiment 126. The kit of Embodiment 125, wherein the human coronavirus is a mutated strain of SARS-CoV-2.

Embodiment 127. The kit of any one of Embodiments 122-126, wherein the inflammatory lung condition or disease caused by the viral infection is hypercytokinaemia, haemophagocytic lymphohistiocytosis, pneumonia, acute respiratory distress syndrome, or systemic inflammatory response syndrome.

Embodiment 128. The kit of Embodiment 127, wherein the inflammatory lung condition or disease caused by the viral infection is acute respiratory distress syndrome.

Embodiment 129. The kit of Embodiment 121, wherein the subject has acute inflammation of the lung.

Embodiment 130. The kit of Embodiment 129, wherein the acute inflammation of the lung is pneumonia or acute respiratory distress syndrome.

Embodiment 131. The kit of Embodiment 121, wherein the subject has an interstitial lung disease.

Embodiment 132. The kit of Embodiment 131, wherein the interstitial lung disease is idiopathic pulmonary fibrosis.

Embodiment 133. The kit of any one of Embodiments 121-132 further comprising administering one or more optional therapeutic agents to the subject.

Embodiment 134. The kit of any one of Embodiments 121-133, comprising administering the hydrochloride salt of racemic 5-[[4-[2-[5-(1-hydroxyethyl)pyridin-2-yl]ethoxy]phenyl]methyl]-1,3-thiazolidine-2,4-dione to the subject.

Embodiment 135. The kit of Embodiment 134, wherein the hydrochloride salt of racemic 5-[[4-[2-[5-(1-hydroxyethyl)pyridin-2-yl]ethoxy]phenyl]methyl]-1,3-thiazolidine-2,4-dione is to be administered to the subject as an oral suspension.

Embodiment 136. The kit of Embodiment 135, wherein about 5 mL to about 25 mL of an oral suspension comprising about 15 mg of the hydrochloride salt of racemic 5-[[4-[2-[5-(1-hydroxyethyl)pyridin-2-yl]ethoxy]phenyl]methyl]-1,3-thiazolidine-2,4-dione per mL is administered to the subject once per day.

Embodiment 137. The kit of any one of Embodiments 121-136, wherein the level of IP-10 is decreased.

Embodiment 138. The kit of any one of Embodiments 121-137, wherein the level of IL-4 is decreased.

Embodiment 139. The kit of any one of Embodiments 121-138, wherein the level of IFNα2 is decreased.

Embodiment 140. The kit of any one of Embodiments 121-139, wherein the level of IFNγ is decreased.

Embodiment 141. The kit of any one of Embodiments 121-140, wherein the level of TNFα is decreased.

Embodiment 142. The kit of any one of Embodiments 121-141, wherein the level of MCP1 is decreased.

Embodiment 143. The kit of any one of Embodiments 121-142, wherein the level of IL-6 is decreased.

Embodiment 144. The kit of any one of Embodiments 121-143, wherein the level of MIP1-α is decreased.

Embodiment 145. The kit of any one of Embodiments 121-144, wherein the level of IP-10, IL-4, IFNα2, IFNγ, TNFα, MCP1, IL-6, or MIP1-α, or a combination thereof, is measured by immunological methods, e.g., ELISA.

Embodiment 146. The kit of any one of Embodiments 121-145, wherein the level of IP-10, IL-4, IFNα2, IFNγ, TNFα, MCP1, IL-6, or MIP1-α, or a combination thereof, is decreased by about 1% to about 100%, e.g., about 5% to about 95%, e.g., about 10% to about 90%, e.g., about 20% to about 85%, e.g., about 30% to about 70%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

Embodiment 147. The kit of Embodiment 146, wherein the level of IP-10 is decreased by about 20% to about 85%.

Embodiment 148. The kit of Embodiments 146 or 147, wherein the level of IL-4 is decreased by about 20% to about 85%.

Embodiment 149. The kit of any one of Embodiments 146-148, wherein the level of IFNα2 is decreased by about 20% to about 85%.

Embodiment 150. The kit of any one of Embodiments 146-149, wherein the level of IFNγ is decreased by about 20% to about 85%.

Embodiment 151. A method, comprising administering a therapeutically effective amount of Compound 1 or Compound 2 to a subject in need thereof, wherein:

(a) the subject has an inflammatory lung condition or disease caused by a viral infection, acute inflammation of the lung, or interstitial lung disease, and

(b) the an inflammatory lung condition or disease caused by a viral infection, acute inflammation of the lung, or interstitial lung disease is characterized as having an increased level of IP-10, IL-4, IFNα2, IFNγ, TNFα, MCP1, IL-6, MIP1-α, IL-12p40, CXCL 9, RANTES, CD45⁺ cells, neutrophils, or T cells, or a combination thereof.

Embodiment 152. The method of Embodiment 151, wherein the subject has an inflammatory lung condition or disease caused by a viral infection.

Embodiment 153. The method of Embodiment 152, wherein the viral infection is a human coronavirus infection, an influenza virus infection, or a HIV virus infection.

Embodiment 154. The method of Embodiment 153, wherein the human coronavirus is HCoV-229E, HCoV-OC43, HCoV-NL63, HCoV-HKU1, SARS-CoV, MERS-CoV, or SARS-CoV-2, or a mutated strain thereof.

Embodiment 155. The method of Embodiment 154, wherein the human coronavirus is SARS-CoV-2.

Embodiment 156. The method of Embodiment 155, wherein the human coronavirus is a mutated strain of SARS-CoV-2.

Embodiment 157. The method of any one of Embodiments 152-156, wherein the inflammatory lung condition or disease caused by the viral infection is hypercytokinaemia, haemophagocytic lymphohistiocytosis, pneumonia, acute respiratory distress syndrome, or systemic inflammatory response syndrome.

Embodiment 158. The method of Embodiment 157, wherein the inflammatory lung condition or disease caused by the viral infection is acute respiratory distress syndrome.

Embodiment 159. The method of Embodiment 151, wherein the subject has acute inflammation of the lung.

Embodiment 160. The method of Embodiment 159, wherein the acute inflammation of the lung is pneumonia or acute respiratory distress syndrome.

Embodiment 161. The method of Embodiment 151, wherein the subject has an interstitial lung disease.

Embodiment 162. The method of Embodiment 161, wherein the interstitial lung disease is idiopathic pulmonary fibrosis.

Embodiment 163. The method of any one of Embodiments 151-162 further comprising administering one or more optional therapeutic agents to the subject.

Embodiment 164. The method of any one of Embodiments 151-163, comprising administering the hydrochloride salt of racemic 5-[[4-[2-[5-(1-hydroxyethyl)pyridin-2-yl]ethoxy]phenyl]methyl]-1,3-thiazolidine-2,4-dione to the subject.

Embodiment 165. The method of Embodiment 164, wherein the hydrochloride salt of racemic 5-[[4-[2-[5-(1-hydroxyethyl)pyridin-2-yl]ethoxy]phenyl]methyl]-1,3-thiazolidine-2,4-dione is administered to the subject as an oral suspension.

Embodiment 166. The method of Embodiment 165, wherein about 5 mL to about 25 mL of an oral suspension comprising about 15 mg of the hydrochloride salt of racemic 5-[[4-[2-[5-(1-hydroxyethyl)pyridin-2-yl]ethoxy]phenyl]methyl]-1,3-thiazolidine-2,4-dione per mL is administered to the subject once per day.

Embodiment 167. The method of any one of Embodiments 151-166, wherein the level of IP-10 is increased.

Embodiment 168. The method of any one of Embodiments 151-167, wherein the level of IL-4 is increased.

Embodiment 169. The method of any one of Embodiments 151-168, wherein the level of IFNα2 is increased.

Embodiment 170. The method of any one of Embodiments 151-169, wherein the level of IFNγ is increased.

Embodiment 171. The method of any one of Embodiments 151-170, wherein the level of TNFα is increased.

Embodiment 172. The method of any one of Embodiments 151-171, wherein the level of MCP1 is increased.

Embodiment 173. The method of any one of Embodiments 151-172, wherein the level of IL-6 is increased.

Embodiment 174. The method of any one of Embodiments 151-173, wherein the level of MIP1-α is increased.

Embodiment 175. The method of any one of Embodiments 151-174, wherein the level of IP-10, IL-4, IFNα2, IFNγ, TNFα, MCP1, IL-6, or MIP1-α, or a combination thereof, is measured by immunological methods, e.g., ELISA.

Embodiment 176. The method of any one of Embodiments 151-175, wherein the level of IP-10, IL-4, IFNα2, IFNγ, TNFα, MCP1, IL-6, or MIP1-α, or a combination thereof, is increased by about 1% to about 100%, e.g., about 5% to about 95%, e.g., about 10% to about 90%, e.g., about 20% to about 85%, e.g., about 30% to about 70%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

Embodiment 177. The method of Embodiment 176, wherein the level of IP-10 is increased by about 20% to about 85%.

Embodiment 178. The method of Embodiments 176 or 177, wherein the level of IL-4 is increased by about 20% to about 85%.

Embodiment 179. The method of any one of Embodiments 176-178, wherein the level of IFNα2 is increased by about 20% to about 85%.

Embodiment 180. The method of any one of Embodiments 176-179, wherein the level of IFNγ is increased by about 20% to about 85%.

Embodiment 181. The method of any one of Embodiments 1-30, wherein the level of IL-12p40 is decreased, e.g., by about 30% to about 90%.

Embodiment 182. The method of any one of Embodiments 1-30 or 181, wherein the level of CXCL 9 is decreased, e.g., by about 30% to about 90%.

Embodiment 183. The method of any one of Embodiments 1-30, 181, or 182, wherein the level of RANTES is decreased, e.g., by about 30% to about 90%.

Embodiment 184. The method of any one of Embodiments 1-30 or 181-183, wherein the level of CD45⁺ cells are decreased, e.g., by about 30% to about 60%.

Embodiment 185. The method of any one of Embodiments 1-30 or 181-184, wherein the level of neutrophils are decreased, e.g., about 40% to about 80%.

Embodiment 186. The composition of any one of Embodiments 31-60, wherein the level of IL-12p40 is decreased, e.g., by about 30% to about 90%.

Embodiment 187. The composition of any one of Embodiments 31-60 or 186, wherein the level of CXCL 9 is decreased, e.g., by about 30% to about 90%.

Embodiment 188. The composition of any one of Embodiments 31-60, 186, or 187, wherein the level of RANTES is decreased, e.g., by about 30% to about 90%.

Embodiment 189. The composition of any one of Embodiments 31-60 or 186-188, wherein the level of CD45⁺ cells are decreased, e.g., by about 30% to about 60%.

Embodiment 190. The composition of any one of Embodiments 31-60 or 186-189, wherein the level of neutrophils are decreased, e.g., by about 40% to about 80%.

Embodiment 191. The Compound 1 or Compound 2 for use of any one of Embodiments 61-90, wherein the level of IL-12p40 is decreased, e.g., by about 30% to about 90%.

Embodiment 192. The Compound 1 or Compound 2 for use of any one of Embodiments 61-90 or 191, wherein the level of CXCL 9 is decreased, e.g., by about 30% to about 90%.

Embodiment 193. The Compound 1 or Compound 2 for use of any one of Embodiments 61-90, 191, or 192, wherein the level of RANTES is decreased, e.g., by about 30% to about 90%.

Embodiment 194. The Compound 1 or Compound 2 for use of any one of Embodiments 61-90 or 191-193, wherein the level of CD45⁺ cells are decreased, e.g., by about 30% to about 60%.

Embodiment 195. The Compound 1 or Compound 2 for use of any one of Embodiments 61-90 or 191-194, wherein the level of neutrophils are decreased, e.g., by about 40% to about 80%.

Embodiment 196. The use of any one of Embodiments 91-120, wherein the level of IL-12p40 is decreased, e.g., by about 30% to about 90%.

Embodiment 197. The use of any one of Embodiments 91-120 or 196, wherein the level of CXCL 9 is decreased, e.g., by about 30% to about 90%.

Embodiment 198. The use of any one of Embodiments 91-120, 196, or 197, wherein the level of RANTES is decreased, e.g., by about 30% to about 90%.

Embodiment 199. The use of any one of Embodiments 91-120 or 196-198, wherein the level of CD45⁺ cells are decreased, e.g., by about 30% to about 60%.

Embodiment 200. The use of any one of Embodiments 91-120 or 196-199, wherein the level of neutrophils are decreased, e.g., by about 40% to about 80%.

### EXAMPLES

### Example 1

### Treatment of COVID-19 Subjects

MIN-102 will be given as a once daily dose for a maximum duration of 28 days to patients with lower respiratory tract infections (LRTI) when presenting with symptoms of pneumonia caused by SARS-CoV-2 infection. The daily dose of MIN-102 will be 180 mg as an oral suspension. This study will recruit male and female hospitalized patients aged >18 years with confirmation of SARS-CoV-2 infection by polymerase-chain reaction (PCR) and LRTI with radiographic evidence of pulmonary infiltrates. Patients will be investigated at regular intervals for incidence of mortality, need for placement in Intensive Care, and time to recovery from symptoms of LRTI based on the following criteria: fever, respiratory rate, oxygen saturation in peripheral blood, need for supplementary oxygen, and severity of cough. When symptoms improve, time to discharge from the hospital will also be determined.

### Example 2

### Cytokine Production in Macrophages, Monocytes and DCs in the Presence of Viral Like Particles Containing the Spike Glycoprotein of SARS-CoV-2

Protocol: SARS-CoV-2 Spike glycoprotein VLPs will be produced by co-transfection of plasmids encoding for the SARS-CoV-2 Spike glycoprotein and different constructs used to produce non-infectious but fusogenic pseudotyped VLP (Ebola VP40-eGFP, Ebola-VP40-Nano-luciferase, and Ebola-VP40-Beta Lactamase). HEK-293T cell will be transfected, and 48 h later supernatants containing viruses will be collected and frozen at −80° C. until use. These viral stocks will be tritiated using Vero E6 and quantified with a VP40 ELISA immunoassay. Macrophages, monocytes, and DCs (APCs; n=3 different cellular donors) will be exposed to VLP cov2-spike/VP40-eGFP and VLP cov2-spike/VP40-Beta Lactamase for 24 h in the absence or presence of leriglitazone. Per condition, 0.8×10⁶ cells will be used in a VF=1 ml in a 12 well plate. Three APCs will be assessed. 24 h post viral exposure, supernatants will be collected, and stored at −80° C. until Luminex assessment. In parallel, cells will be also collected and assed by FACS to measure:

1) Viral uptake by eGFP

2) Viral fusion by beta-lactamase

3) Up-regulation of activation markers (HLA-DR, CD83, CD86, Siglec-1, DC-SIGN).

Supernatants will be assessed for cytokine release and compared to mock-treated cells using Luminex technology.

### Example 3

### Immunomodulatory Effect of MIN-102 on Cytokine Production in Macrophages in the Presence of SARS-CoV-2

### Material & Methods

SARS-CoV-2 clinical isolate grown in Vero E6 cells and already sequenced was titrated in Vero E6 cells to stablish “Tissue Culture Infectious Doses that kills 50% of the cells” (TCID50) and quantified with a SARS-CoV-2 nucleoprotein ELISA immunoassay (Sino Biologicals).

Macrophages derived from monocytes (negative selection) from 3 donors were cultured as described in Pino et al., Retrovirology 12:37 (2015) and exposed to SARS-CoV-2 clinical isolate at an MOI/TCID50 of 1-2 for 24 h or left untreated in the absence or presence of MIN-102.

MIN-102 (leriglitazone) was added at two different time points: 1) Pre-treatment (48 hours prior infection and at infection), and, 2) Therapeutic (at infection). Three different concentrations were assayed: 0.5 μM, 2 μM, and 5 μM, following resuspension. Control cells included the DMSO vehicle used for resuspension at 5 μM. 0.3×10⁶ cells in a Vf of 330 μl per well in duplicates were plated in a 24 well plate. The following conditions were assayed per donor:

NO VIRUS

Vehicle at 5 μM with DMSO 10%;

MIN-102 at 500 nM (0.1 mM DMSO 10%) for therapeutic and pretreatment; and

MIN-102 at 5 μM (0.1 mM DMSO 10%) for therapeutic and pretreatment.

SARS-CoV-2 VIRUS

Vehicle at 5 μM with DMSO 10%;

MIN-102 at 500 nM (0.1 mM DMSO 10%) for therapeutic and pretreatment;

MIN-102 at 2 μM (0.1 mM DMSO 10%) for therapeutic and pretreatment; and

MIN-102 at 5 μM (0.1 mM DMSO 10%) for therapeutic and pretreatment.

Cells and supernatant (600 μl) were collected at 24 h post infection. Cells were lysed with lysis buffer for ELISA and harvested by centrifugation at 300 g for 5 min to collect supernatant for ELISA. Cells were also assayed for viability using the Cell titter Glo luminescence assay. Supernatant and lysed cells were stored at −80° C. until processing.

Collected supernatants fixed with formaldehyde (Dowall et al., J. Immunol. Methods 348(1-2):30-35 (2009)) to inactivate virus were assessed for cytokine release and compared to mock-treated cells using Luminex technology. The cytokine panel included the following cytokines: IL-2, IL-7, IL-6, IL-4, interferon-7, inducible protein 10 (IP-10), monocyte chemoattractant protein 1 (MCP1), macrophage inflammatory protein 1-α (MIP1-α), tumor necrosis factor-α (TNFα), and interferon alpha-2 (IFNα2).

### Results

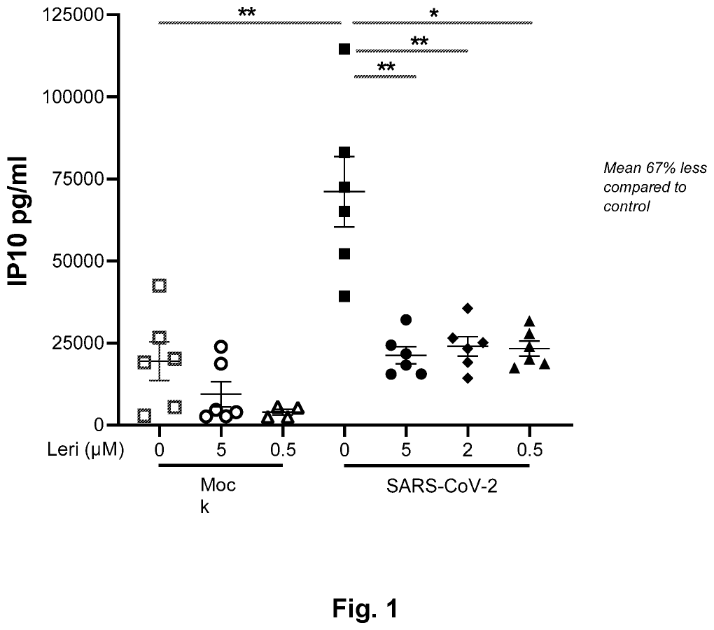

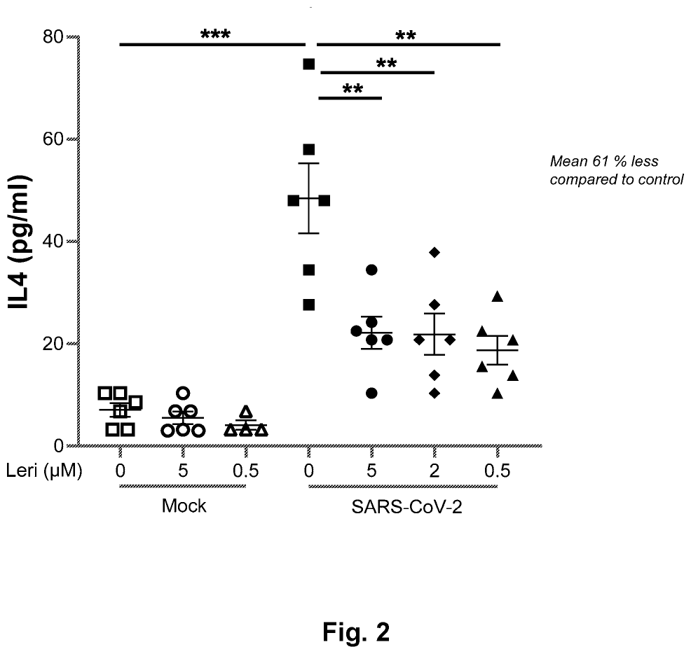

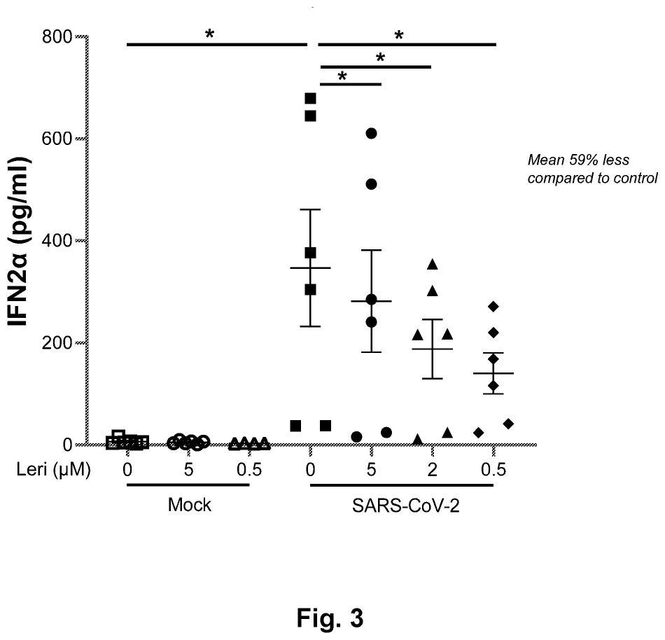

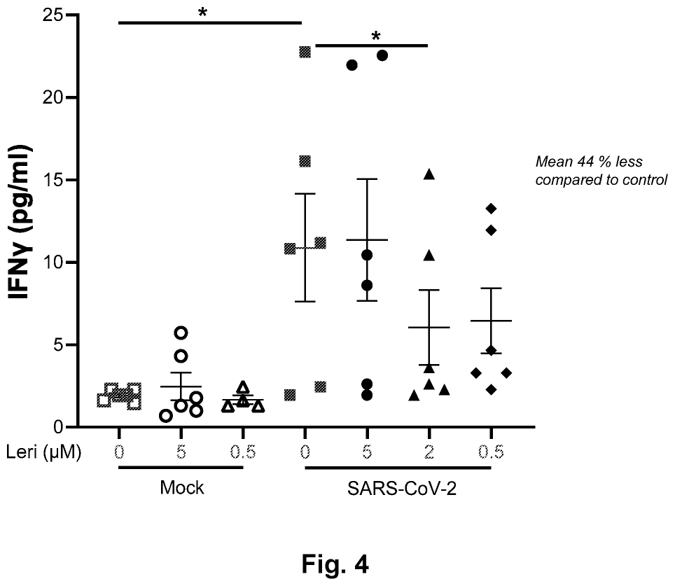

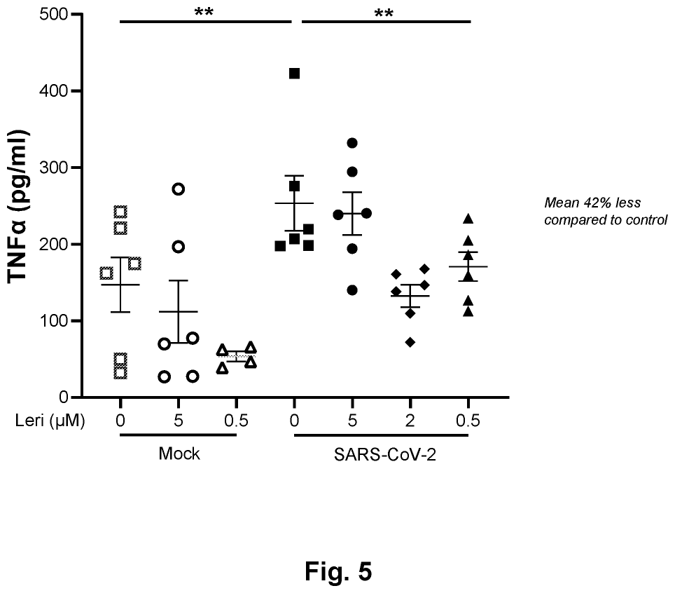

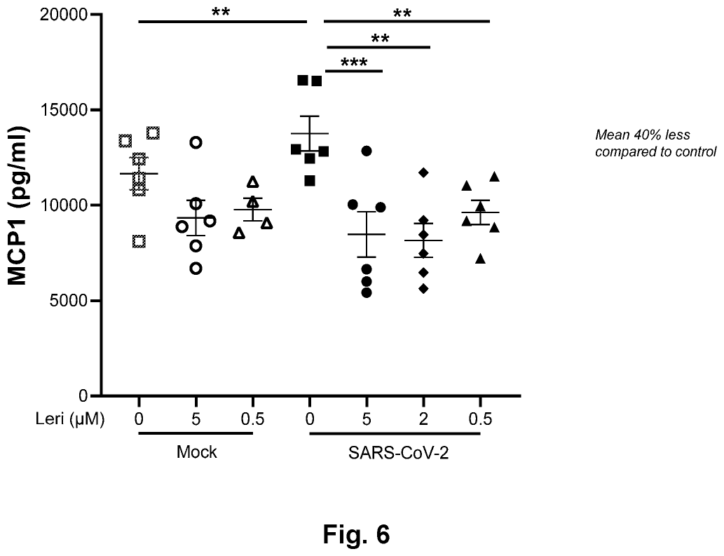

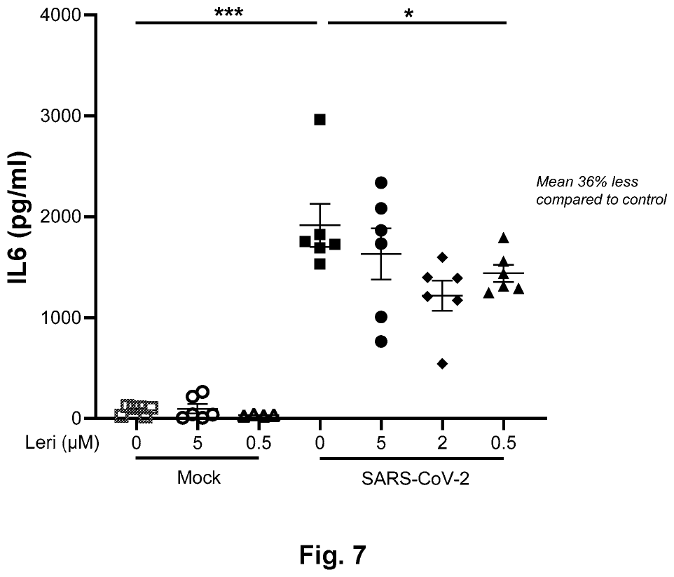

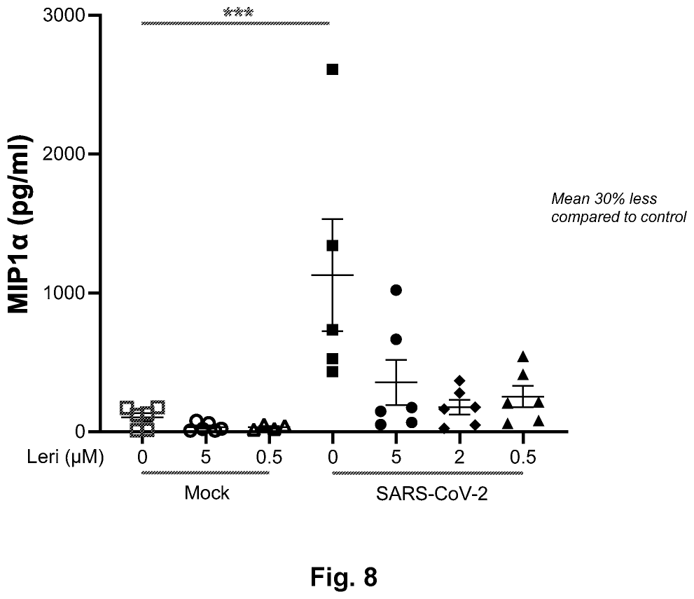

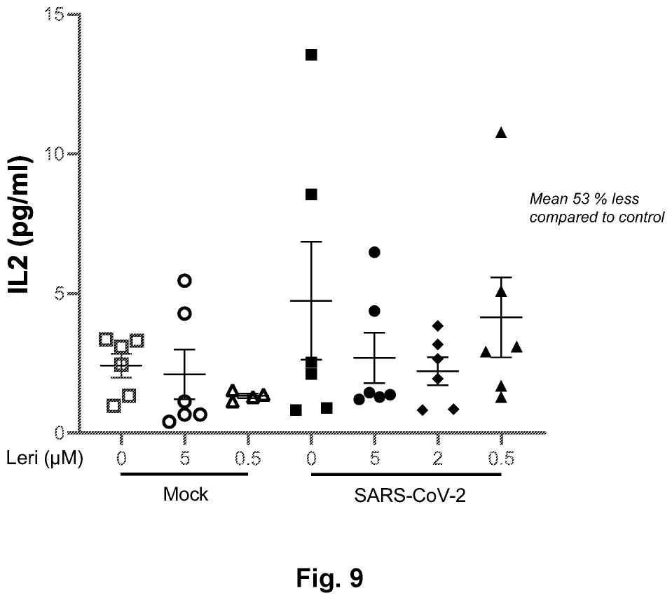

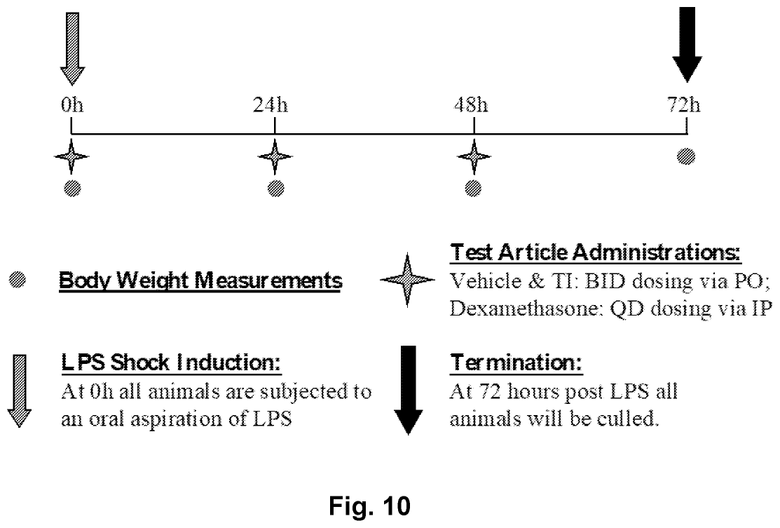

Several cytokines were measured in the supernatant of mock-treated macrophages, and compared to SARS-CoV-2 exposed cultures 24 h post-infection using Luminex technology. Upon SARS-CoV-2 exposure, macrophages released higher levels of IP-10, IL-4, IFNα2, IFNγ and IL-6 (FIGS. 1-10). The trend of increase was also observed for other cytokines such as TNFα and MIP1-α.

There was a decrease of those cytokines up-regulated by SARS-CoV-2 in the presence of MIN-102, and that effect was mostly observed for the therapeutic condition. A significant reduction of cytokines (ranging from a mean of 67% to a 36% reduction of cytokine secretion) was observed for IP-10, IL4, IFNα2, IFNγ, TNFα, MCP1 and IL6. For MIP1-α, reduction reached 90%. No effect on IL2 and IL7 secretion was detected.

The results of this experiment indicate that MIN-102 exerts an effect on macrophages in the presence of SARS-CoV-2, aiding to decrease several cytokines implicated in ARDS complication. Del Valle et al., Nat. Med. 26:17 (2020). This effect was particularly significant at the therapeutic condition and at a concentration of 2 μM.

The therapeutic benefit of downregulating IP-10, IFNα2, IFNγ, TNFα, IL-6, MCP1 and MIP1-α in COVID-19 patients by administration of MIN-102 can be summarized as follows:

IP-10: Increased in patients that went onto develop worse disease. Takahashi et al., Nature 588:315-320 (2020).

IFNα2 and IFNγ: Interferons are potent antivirals that may aid to control viral replication in SARS-COV-2 infected individuals. However, viral replication decreases over time, and at later stages of disease progression, these IFNs may contribute to immune activation. Moreover, as the viral receptor ACE2 is an interferon stimulated gene (Ziegler et al., Cell 181(5):1016-1035.e19 (2020)), controlling interferons may aid to decrease the expression of this receptor. While critical COVID-19 patients have impaired IFN responses (Hadjadj et al., Science 369(6504):718-724 (2020)), treatment with IFNγ has not demonstrated any benefit in the large clinical trial of the WHO. Pan et al., N Engl J Med, 2020 Dec. 2: NEJMoa2023184. Moreover, dexamethasone, which is the only treatment that has reduced mortality in patients requiring respiratory support (Horby et al., N Engl J Med, 2020 Jul. 17: NEJMoa2021436) also inhibits type I interferons (Flammer et al., Mol. Cell. Biol. 30(19):4564-4574 (2010)) and type III interferons. Hu et al., J. Immunol. 170(9):4833-4839 (2003).

TNFα and IL-6: Serum levels of these cytokines are independent and significant predictors of disease severity and death. Del Valle et al., Nat. Med. 26:17 (2020).

MCP1 and MIP1-α: Compared with non-ICU patients, ICU patients had higher plasma levels of these two cytokines. Huang et al., The Lancet. 395(10223):497-506 (2020).

### Example 4

### Evaluation of MIN-102 and M3 in LPS Induced Acute Lung Injury Model in Mouse

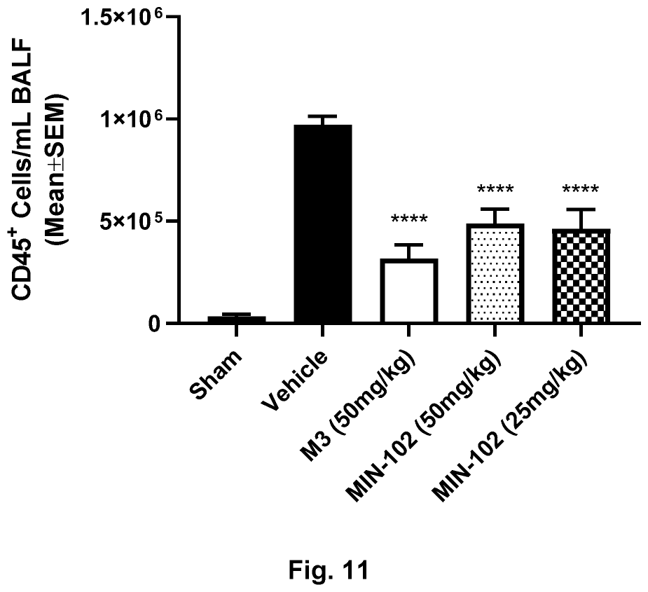

Table 2 summarizes the groups tested in the LPS induced acute lung injury (ALI) model study in mice. The study schematic is shown in FIG. 11.

TABLE 2

| | | | | | | Dosing | |
|---|---|---|---|---|---|---|---|
| Group | Group | Group | Disease | | Dose | Volume | |
| No. | Size | Description | Induction | Route | (mg/kg) | (mL/kg) | Regiment |
| 1 | N = 5 | Sham (Saline) | 50 μL of | N/A | N/A | N/A | N/A |
| | | | saline OA | | | | |
| | | | on Day 0 | | | | |
| 4 | N = 10 | Vehicle | 50 μL of | PO | N/A | 10 | BID from |
| 5 | N = 10 | M3 | LPS OA | PO | 50 | 10 | Day −1 to 2, |
| 6 | N = 10 | MIN-102 | on Day 0 | PO | 50 | 10 | 6-8 hrs |
| 7 | N = 10 | MIN-102 | | PO | 25 | 10 | apart and |
| | | | | | | | QD on Day 3 |
| | | | | | | | 1 h before |
| | | | | | | | euthanasia |

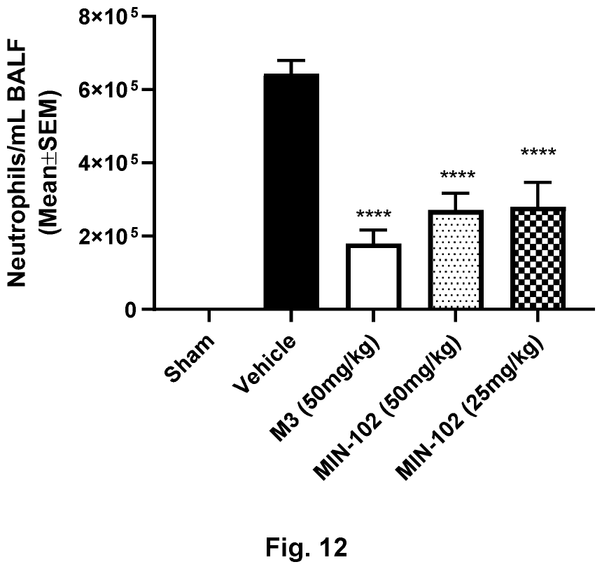

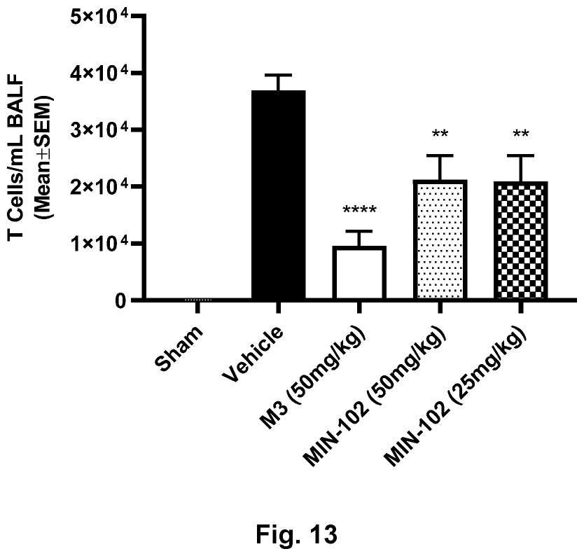

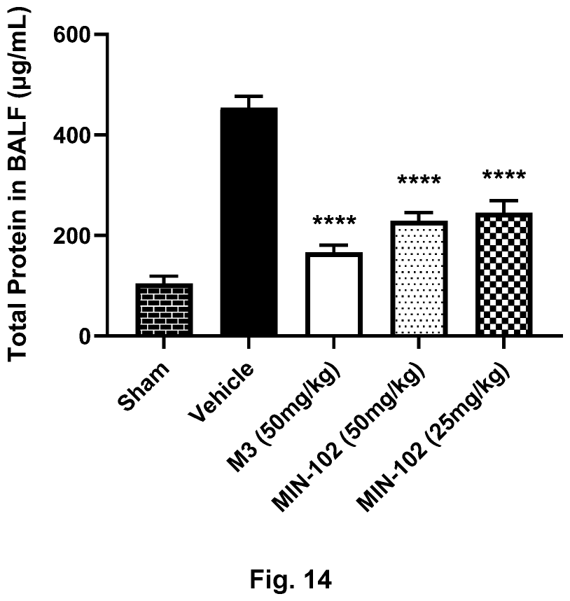

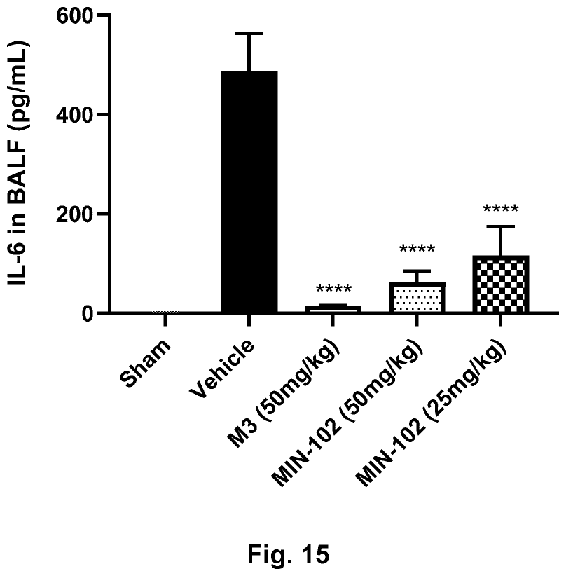

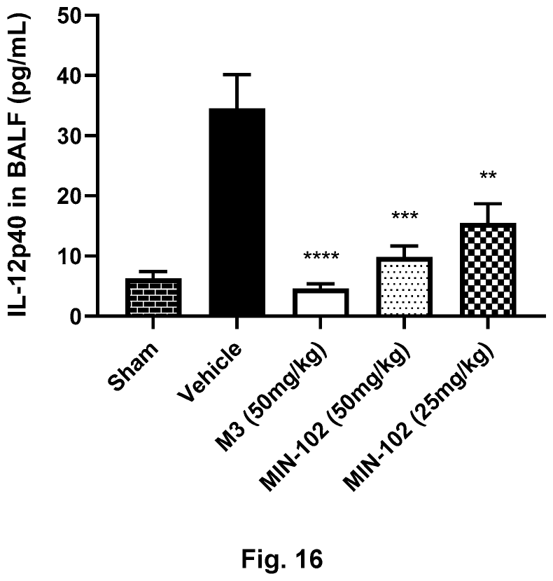

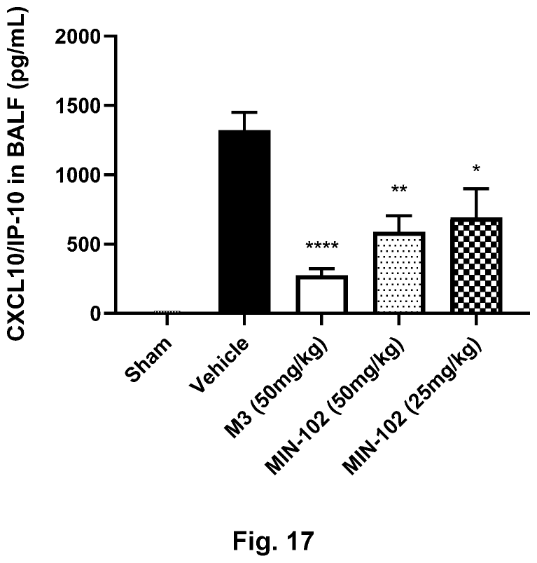

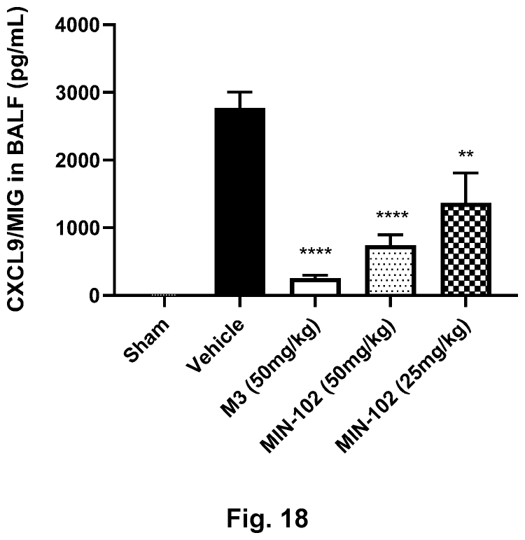

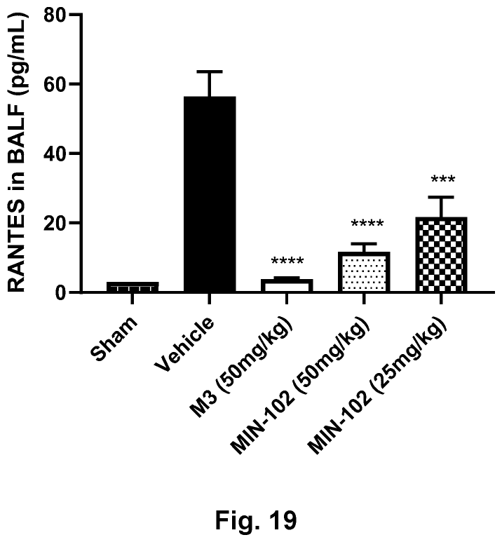

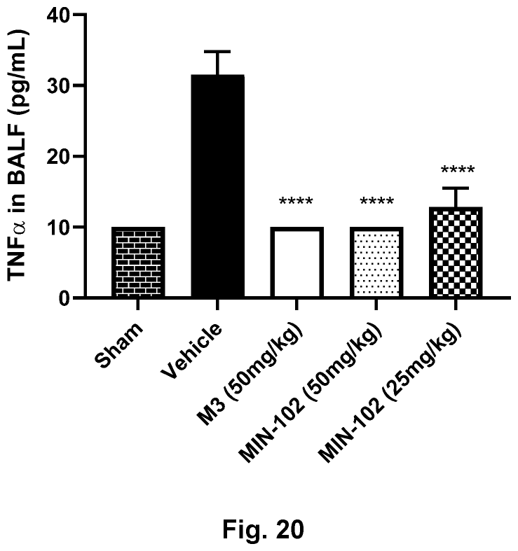

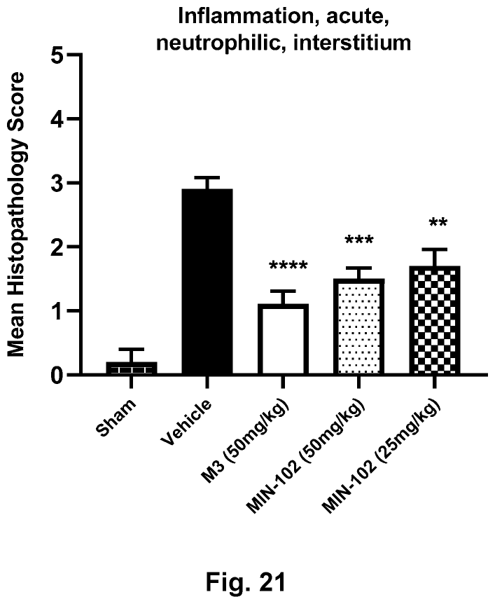

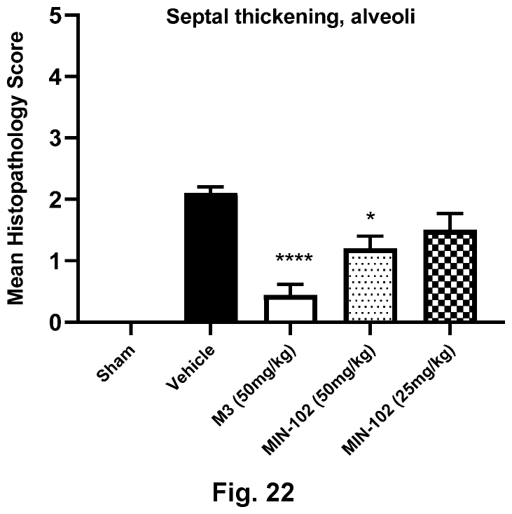

Briefly, C57Bl/6 7-8 week old mice were exposed to lipopolysaccharide (LPS) oral aspiration (5 mg/Kg). The animals were treated orally with either vehicle, M3 (50 mg/Kg, or MIN-102 at different doses 25 and 50 mg/Kg) bid from Day −1 to Day 2, 6-8 h apart and QD on Day 3 1 h before euthanasia. On Day 3 protein content, cytokine assessment and leukocyte count by FACS analysis were performed in the bronchoalveolar lavage fluid (BALF). The results are shown in FIGS. 11-20, and Tables 3 and 4. The lungs were collected, embedded and processed for H&E histology. The results are shown in FIGS. 21 and 22.

TABLE 3

| | CD45+ | | Neutrophils | | T cells | | | |
|---|---|---|---|---|---|---|---|---|
| | Mean | % | Mean | | Mean | | Total Protein in BALF | |
| | (cells/ml | Decrease | (cells/ml | % Decrease | (cells/ml | % Decrease | Mean | % Decrease |
| Treatment | BALF) | vs vehicle | BALF) | vs vehicle | BALF) | vs vehicle | (ug/ml) | vs vehicle |
| Vehicle | 971703 | | 643501 | | 36941 | | 454.3 | |
| M3 | 317269 | 67.35 | 179793 | 72.06 | 9554 | 74.14 | 167 | 63.24 |
| 50 mg/kg | | | | | | | | |
| MIN-102 | 487506 | 49.83 | 271223 | 57.85 | 21245 | 42.49 | 229.4 | 49.50 |
| 50 mg/kg | | | | | | | | |
| MIN-102 | 462941 | 52.36 | 280504 | 56.41 | 20898 | 43.43 | 245.3 | 46.00 |
| 25 mg/kg | | | | | | | | |

TABLE 4

| | IL-6 | | IL-12p40 | | IP-10 | | MIG | | RANTES | | TNF | |
|---|---|---|---|---|---|---|---|---|---|---|---|---|
| | | % | | % | | % | | % | | % | | % |
| | Mean | Decrease | Mean | Decrease | Mean | Decrease | Mean | Decrease | Mean | Decrease | Mean | Decrease |
| Treatment | (pg/ml) | vs vehicle | (pg/ml) | vs vehicle | (pg/ml) | vs vehicle | (pg/ml) | vs vehicle | (pg/ml) | vs vehicle | (pg/ml) | vs vehicle |
| Vehicle | 487.7 | | 34.51 | | 1320 | | 2767 | | 56.41 | | 31.51 | |
| M3 | 15.29 | 96.86 | 4.586 | 86.71 | 1045 | 79.17 | 256.2 | 90.75 | 3.838 | 93.19 | 10 | 68.26 |
| 50 mg/kg | | | | | | | | | | | | |
| MIN-102 | 62.78 | 87.13 | 9.868 | 71.41 | 731.5 | 55.42 | 741.7 | 73.18 | 11.71 | 79.24 | 10 | 68.26 |
| 50 mg/kg | | | | | | | | | | | | |
| MIN-102 | 116.2 | 76.17 | 15.48 | 55.14 | 627.6 | 47.55 | 1369 | 50.52 | 21.68 | 61.57 | 12.85 | 59.19 |
| 25 mg/kg | | | | | | | | | | | | |

MIN-102 decreased the total number of leukocytes (CD45 positive cells) vs vehicle group by 50% in all doses and neutrophils (maximal reduction around 58%). T cells (maximal reduction around 47%). The total protein content in the BALF was also decreased from 30 to 50% in a dose dependent manner. Likewise, M3 reduced total number of leukocytes by 67%, neutrophils by 72%, B cells by 52%, T cells by 74%, and the protein content by 63%.

Cytokine analysis revealed the effect of both MIN-102 and M3 in the reduction of the cytokine and chemokine secretion in the BALF (IL-6, IL12p40; IP-10, MIG, RANTEs and TNF-alpha) from 50 to 90% mostly in a dose dependent manner.

MIN-102 and M3 reduce the recruitment and activation of neutrophils and T cells thereby preventing LPS-induced ALI.

The histology study shows improvement from treatment of M3 or MIN-102. For example, there is a reduction in mononuclear cell infiltration in the perivascular/peribronchiolar space (40 and 31%) and decreases in neutrophil recruitment in the interstitium (62 and 48%) and the alveoli (64 and 53%). See FIG. 21. In addition, the septal thickening in the alveoli was reduced (79 and 43%) for M3 and MIN-102, respectively. See FIG. 22.

Having now fully described the compounds, methods, kits, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications and publications cited herein are fully incorporated by reference herein in their entirety.

1. A method of treating an inflammatory lung condition or disease caused by a viral infection, acute inflammation of the lung, or interstitial lung disease in a subject in need thereof, the method comprising administering to the subject a therapeutically effective amount of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof.

2. The method of claim 1 for treating an inflammatory lung condition or disease caused by a viral infection in a subject in need thereof.

3. The method of claim 2, wherein the viral infection is a human coronavirus infection, an influenza virus infection, or a HIV virus infection.

4. (canceled)

5. The method of claim 3, wherein the human coronavirus is SARS-CoV-2 or a mutated strain of SARS-CoV-2.

6. (canceled)

7. The method of claim 2, wherein the inflammatory lung condition or disease caused by the viral infection is hypercytokinaemia, haemophagocytic lymphohistiocytosis, pneumonia, acute respiratory distress syndrome, or systemic inflammatory response syndrome.

8. (canceled)

9. The method of claim 1 for treating acute inflammation of the lung in a subject in need thereof.

10. The method of claim 9, wherein the acute inflammation of the lung is caused by a bacterial infection.

11. The method of claim 9, wherein the acute inflammation of the lung is pneumonia or acute respiratory distress syndrome.

12. The method of claim 1 for treating interstitial lung disease in a subject in need thereof.

13. The method of claim 12, wherein the interstitial lung disease is idiopathic pulmonary fibrosis.

14. The method of claim 1 further comprising administering one or more optional therapeutic agents to the subject.

15. The method of claim 1, comprising administering the hydrochloride salt of racemic 5-[[4-[2-[5-(1-hydroxyethyl)pyridin-2-yl]ethoxy]phenyl]methyl]-1,3-thiazolidine-2,4-dione to the subject.

16-59. (canceled)

60. A kit comprising 5-[[4-[2-[5-(1-hydroxyethyl)pyridin-2-yl]ethoxy]phenyl] methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof, in a container and instructions for administering 5-[[4-[2-[5-(1-hydroxyethyl)pyridin-2-yl]ethoxy]phenyl] methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof, to a subject having an inflammatory lung condition or disease caused by a viral infection, acute inflammation of the lung, or interstitial lung disease.

61. The kit of claim 60 for treating an inflammatory lung condition or disease caused by a viral infection in a subject in need thereof.

62. The kit of claim 61, wherein the viral infection is a human coronavirus infection, an influenza virus infection, or a HIV virus infection.

63-65. (canceled)

66. The kit of claim 61, wherein the inflammatory lung condition or disease caused by the viral infection is hypercytokinaemia, haemophagocytic lymphohistiocytosis, pneumonia, acute respiratory distress syndrome, or systemic inflammatory response syndrome.

67. (canceled)

68. The kit of claim 60 for treating acute inflammation of the lung in a subject in need thereof.

69. (canceled)

70. (canceled)

71. The kit of claim 60 for treating interstitial lung disease in a subject in need thereof.

72. (canceled)

73. (canceled)

74. A method for decreasing the level of IP-10, IL-4, IFNα2, IFNγ, TNFα, MCP1, IL-6, MIP1-α, IL-12p40, CXCL 9, RANTES, CD45⁺ cells, T cells, or neutrophils, or a combination thereof, in a subject in need thereof, the method comprising administering to the subject a therapeutically effective amount of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof, wherein the subject has an inflammatory lung condition or disease caused by a viral infection, acute inflammation of the lung, or interstitial lung disease.

75-77. (canceled)

78. A method, comprising administering a therapeutically effective amount of 5-[[4-[2-[5-(1-hydroxyethyl)pyridin-2-yl]ethoxy]phenyl]methyl]-1,3-thiazolidine-2,4-dione, or a pharmaceutically acceptable salt thereof, or 5-[[4-[2-[5-acetylpyridin-2-yl]ethoxy]phenyl]methyl]-1,3-thiazolidine-2,4-dione, or a pharmaceutically acceptable salt thereof, to a subject in need thereof, wherein:
(a) the subject has an inflammatory lung condition or disease caused by a viral infection, acute inflammation of the lung, or interstitial lung disease, and
(b) the inflammatory lung condition or disease caused by a viral infection, acute inflammation of the lung, or interstitial lung disease is characterized as having an increased level of IP-10, IL-4, IFNα2, IFNγ, TNFα, MCP1, IL-6, MIP1-α, IL-12p40, CXCL 9, RANTES, CD45⁺ cells, T cells, or neutrophils, or a combination thereof.
